(19) United States
(12) Patent Application Publication
(10) Pub. No.: US 2019/0225670 A1
(43) Pub. Date: Jul. 25, 2019

(54) COMPOUNDS AND METHODS FOR ACTIVATING TIE2 SIGNALING

(71) Applicants: ASCLEPIX THERAPEUTICS, LLC, Baltimore, MD (US); The Johns Hopkins University, Baltimore, MD (US)

(72) Inventors: Niranjan B. Pandey, Baltimore, MD (US); Adam Mirando, Baltimore, MD (US); Aleksander S. Popel, Baltimore, MD (US); Jordan J. Green, Baltimore, MD (US)

(21) Appl. No.: 16/336,777

(22) PCT Filed: Oct. 4, 2017
(86) PCT No.: PCT/US17/55055

§ 371 Date: Mar. 26, 2019

Related U.S. Application Data
(60) Provisional application No. 62/403,786, filed on Oct. 4, 2016.

(51) Int. Cl. C07K 14/78; A61P 7/10; A61K 38/39; A61P 9/10; A61K 47/69

(52) U.S. Cl. CPC C07K 14/78; A61P 7/10; A61K 38/00; A61P 9/10; A61K 47/6937; A61K 38/39

(57) ABSTRACT

The present invention in various aspects and embodiments, involves methods for treating Tie2-related vascular permeability by administering one or more collagen IV-derived biomimetic peptides and involves compositions for treating Tie2-related vascular permeability comprising one or more collagen IV-derived biomimetic peptides. Such peptides can promote the Tie2 agonist activities of Angiopoietin 2 (Ang2), thereby stabilizing vasculature and/or lymphatic vessels.

### CROSS-REFERENCE TO RELATED APPLICATIONS

This application claims priority to U.S. Provisional Patent Application No. 62/403,786, filed Oct. 4, 2016, the entire contents of which is incorporated herein by reference.

### STATEMENT OF RIGHTS TO INVENTIONS MADE UNDER FEDERALLY SPONSORED RESEARCH

This invention was made with government support under R01CA138264 and 1R21EY026148 by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Oct. 1, 2017, is named ASX-002PC-SequenceListing_ST25.txt and is 13,204 bytes in size.

### BACKGROUND

The Tie2 receptor tyrosine kinase signaling pathway, and its ligands Angiopoietin1 (Ang1) and Angiopoietin2 (Ang2), regulate vascular permeability. Vascular permeability is compromised in patients with macular edema including patients with retinal vein occlusion (RVO), diabetic macular edema (DME), wet age-related macular degeneration (wet AMD), and background diabetic retinopathy (DR), as well as many other conditions. Tie2 may also regulate lymphatic vessel integrity especially during inflammation.

Ang1 binds Tie2 and stimulates phosphorylation and downstream signaling stabilizing blood vessels. Ang2 competes with Ang1 for Tie2 binding reducing the phosphorylation of Tie2, and thus it acts as an endogenous Tie2 antagonist. Ischemic or hypoxic retina produces high levels of Ang2, and Ang2 levels, like that of VEGF levels, are increased in the eyes of DME patients. Ang2 increases the responsiveness of retinal vessels to VEGF and promotes vascular leakage and neovascularization.

Ang2 may also act as a weak agonist of Tie2 especially when Ang1 levels are low. Specifically, exogenous Ang2 activates Tie2 and the promigratory, prosurvival PI3K/Akt pathway in endothelial cells (ECs) but with less potency and lower affinity than exogenous Ang1. ECs produce Ang2 but not Ang1. This endogenous Ang2 maintains Tie2, phosphatidylinositol 3-kinase, and Akt activities, and it promotes EC survival, migration, and tube formation.

Restoration of Tie2 activation could provide benefit in conditions associated with edema and vascular integrity, including macular edema, DME, and other conditions.

### SUMMARY OF THE INVENTION

In various aspects and embodiments, the invention provides methods and compositions for treating Tie2-related vascular permeability, by administering one or more collagen IV-derived biomimetic peptides. Such peptides can promote the Tie2 agonist activities of Angiopoietin 2 (Ang2), thereby stabilizing vasculature and/or lymphatic vessels. In various embodiments, the biomimetic peptide can be delivered for treatment of conditions such as macular edema, wet AMD, and treatment or prevention of tumor growth or metastasis, among others. In some embodiments, the condition is refractory or only partially-responsive to VEGF blockade therapy or kinase inhibitor therapy. For example, the biomimetic peptide may be administered after unsuccessful VEGF blockade therapy, that is, where significant reductions in angiogenesis, lymphangiogenesis, and/or edema were not observed. In some embodiments, the peptide is administered as an alternative to, or in combination with, VEGF blockade therapy. By activating Tie2 signaling, the biomimetic peptides or peptide agents provide therapeutic benefits that may not be observed with VEGF-blockage therapy, or with VEGF blockade therapy alone.

Collagen IV-derived biomimetic peptides are derived from the α5 fibril of type IV collagen. The peptides may target α5β1 and αVβ3 integrins in some embodiments, and may inhibit signaling through multiple receptors, including vascular endothelial growth factor receptor (VEGFR), hepatocyte growth factor receptor (HGFR), insulin-like growth factor receptor (IGFR), and epidermal growth factor receptor (EGFR). As disclosed herein, collagen IV-derived biomimetic peptides further promote Tie2 agonist activities of Angiopoietin 2, thereby stabilizing vasculature and/or lymphatic vessels.

The biomimetic peptide or peptide agent may be formulated for local delivery to affected tissues or by systemic delivery, for example, using a variety of pharmaceutically acceptable carriers. In some embodiments, the peptide is formulated with a polymeric nanoparticle or microparticle carrier, which may comprise a material having one or more degradable linkages. The peptide may be conjugated to the surface of the particles, or may be encapsulated within the particles for sustained release. In some embodiments the particles comprise poly(lactic-co-glycolic acid) polyethylene glycol (PLGA-PEG) block copolymers of tunable size which are covalently linked to the biomimetic peptide. The particles may be designed to provide desired pharmacodynamic advantages, including circulating properties, biodistribution, degradation kinetics, including the tuning of surface properties.

In some embodiments, the nanoparticles further comprise an encapsulated active agent, for treatment of a Tie2-related condition. For example, the particle may be a microparticle that encapsulates an effective amount of a biomimetic peptide to provide a long acting drug depot or to provide a sustained release of the biomimetic peptide or peptide agent.

In certain aspects, the invention provides a method for preventing or treating a condition involving Tie-2-related vascular permeability or lymphatic vessel integrity in a patient. The method comprises administering the collagen IV-derived biomimetic peptide, or particle formulation thereof, to the patient in an amount effective to reduce Tie2-dependent vascular permeability or lymphatic vessel integrity. Restoration of Tie2 activation provides therapeutic benefit in conditions associated with edema or vascular permeability, including macular edema, diabetic macular edema (DME), and other conditions, including conditions characterized by acute or chronic inflammation. Tie2-related conditions include diabetic macular edema, retinal vein occlusion, wet AMD, background diabetic retinopathy, cancer (including for reducing, slowing or preventing tumor growth or metastasis), influenza, hemorrhagic fever, cerebral malaria, Alzheimer's disease, acute respiratory distress syndrome, pulmonary edema, asthma, Respiratory Syncytial Virus, COPD, SARS, pneumonia, sepsis among others.

Other aspects and embodiments of the invention will be apparent from the following detailed description.

### BRIEF DESCRIPTION OF THE DRAWINGS

FIG. 1 shows that AXT107 (identified in FIG. 1 as SP2043) promotes the agonist activity of Ang2 to activate the Tie2 signaling pathway.

FIG. 2A shows western blots of microvascular endothelial cell (MEC) lysates treated with Ang2 and AXT107 showing phosphorylation of Tie2 and the downstream effectors STAT3, Akt, and Erk1/2, with GAPDH as a loading control. FIG. 2B shows graphs illustrating densitometric analyses of the western blots disclosed in FIG. 2A and adjusted for loading control and presented relative to Ang2-alone control. One-way ANOVA; N=3; *, *** p≤0.05 and 0.001, respectively, relative to Ang2-alone control.

FIG. 3A shows immunofluorescence images of MEC monolayers treated with 0.1% BSA in PBS (left column) or 200 ng/ml Ang2 (right column) for fifteen minutes at varying concentrations of AXT107 and stained for phospho-Tie2 (Y992) (green) and DAPI (blue). White arrows indicate junctional Tie2. FIG. 3B includes western blots of MEC lysates treated with various growth factors and 100 μM AXT107 or DMSO vehicle and fractioned into Triton X-100-soluble and Triton X-100-insoluble pools. FIG. 3C is a graph illustrating densitometric analyses of the western blots disclosed in FIG. 3B; each bar represents the percent change of AXT107-treated samples relative to the corresponding vehicle control of the same growth factor and separated by soluble (left bars) and insoluble (right bars). FIG. 3D is a representative image (n=3) of western blots of Triton X-100-fractionated lysates which were immunoprecipitated for Tie2 and blotted for phospho-Tie2 (top) or for total Tie2 (bottom).

FIGS. 4A and 4C shows western blots of MEC lysates treated with various growth factors and 100 μM AXT107 or DMSO vehicle and fractioned into Triton X-100-soluble and Triton X-100-insoluble pools and immunoblotted for integrin α5 (A) or immunoblotted for integrin β₁ (C). FIGS. 4B and 4D are graphs illustrating densitometric analyses of bands from, respectively, FIG. 4A and FIG. 4C; each bar represents the percent change of AXT107-treated samples relative to the corresponding vehicle control of the same growth factor and separated by soluble (left bars) and insoluble (right bars). FIG. 4E shows western blots of Triton X-100-fractionated lysates which were immunoprecipitated for integrin α₅ and blotted for integrin as (top) or for integrin β₁ (bottom). FIG. 4F shows photomicrographs of representative images of a Duolink™ assay showing interactions between α₅ and β₁ integrins in MEC monolayers treated with vehicle or with 100 μM AXT107 and FIG. 4G is a graph quantifying the interactions per arbitrary area. FIGS. 4H and 41 show representative images (n=3) of western blots of Triton X-100-fractionated lysates which were immunoprecipitated for Tie2 and blotted for α5 integrin; cells in FIG. 4H were treated with 200 ng/ml of Ang2 and cells in FIG. 4I were not treated with a growth factor. N=3.

FIG. 5A shows a representative western blot of VE-Cadherin from MEC monolayers treated with 200 ng/ml Ang2 for three hours at various concentrations of AXT107; GAPDH is included as a loading control. FIG. 5B shows photomicrographs of immunofluorescence images of MEC monolayers treated with 200 ng/ml Ang2 and various concentrations of AXT107 that have been stained with antibodies targeting VE-cadherin (green), F-actin (red), and DAPI (blue) and with merged regions shown in yellow; arrows indicate representative regions showing transition of VE-cadherin distribution. FIG. 5C is a graph quantifying the average area of F-actin coverage per cell; one-way ANOVA; N=3; *, ** p≤0.05 and 0.01, respectively, relative to Ang2 alone control. FIG. 5D shows representative western blot images of lysates from MECs treated with 200 ng/ml Ang2 and various concentrations of AXT107 blotted against pMLC2 and with GAPDH as a loading control. FIG. 5E is a graph showing a densitometric analysis of the data shown in FIG. 5D; one-way ANOVA; N=3; *** p≤0.001 relative to Ang2-alone control. FIG. 5F is a schematic of transendothelial permeability assay described in Example 5. FIG. 5G is a graph showing quantification of FITC-Dextran (40 kDa) migration across MEC monolayers plated on semipermeable substrates following treatment with growth factors and AXT107, where indicated. Student's two-tailed t-test; N≥7; * p≤0.05.

FIG. 6 includes a model for AXT107-mediated activation of Tie2.

### DETAILED DESCRIPTION

In various aspects and embodiments, the invention provides methods and compositions for treating Tie2-related vascular or lymphatic vessel permeability, by administering one or more collagen IV-derived biomimetic peptide(s). Such peptides can promote the Tie2 agonist activities of Angiopoietin 2 (Ang2), thereby stabilizing vasculature and/or lymphatic vessels.

Collagen IV-derived biomimetic peptides are derived from the α5 fibril of type IV collagen. Exemplary peptides comprise, consist of, or consist essentially of the amino acid sequence LRRFSTAPFAFIDINDVINF (SEQ ID NO:1), or derivatives thereof. The peptides may target α5β1 and αVβ3 integrins, and inhibit signaling through multiple receptors, including vascular endothelial growth factor receptor (VEGFR), hepatocyte growth factor receptor (HGFR), insulin-like growth factor receptor (IGFR), and epidermal growth factor receptor (EGFR). As disclosed herein, collagen IV-derived biomimetic peptides further promote Tie2 agonist activities of Angiopoietin 2, thereby stabilizing vasculature and/or lymphatic vessels.

Collagen IV-derived biomimetic peptides include those described in U.S. Pat. No. 9,056,923, which is hereby incorporated by reference in its entirety. For example, peptides in accordance with the following disclosure include peptides comprising the amino acid sequence LRRFSTXPXXXXNINNVXNF (SEQ ID NO:2), where X is a standard amino acid or non-genetically encoded amino acid. In some embodiments, X at position 7 is M, A, or G; X at position 9 is F, A, Y, or G; X at position 10 is M, A, G, D-Alanine (dA), or norleucine (Nle); X at position 11 is F, A, Y, G, or 4-chlorophenylalanine (4-ClPhe); X at position 12 and position 18 are independently selected from 2-Aminobutyric acid (Abu), G, S, A, V, T, I, L, or Allylglycine (AllylGly). In various embodiments, the peptide contains about 30 amino acids or less, or about 25 amino acids of less, or about 24 amino acids, or about 23 amino acids, or about 22 amino acids, or about 21 amino acids, or about 20 amino acids. In still other embodiments, one, two, three, four, or five amino acids of SEQ ID NO:2 are deleted.

In some embodiments, the peptide comprises the amino acid sequence LRRFSTAPFAFIDINDVINF (SEQ ID NO:3), or derivative thereof. The peptide of SEQ ID NO:3 is also referred to as AXT107 or as SP2043. Derivatives of the peptide of SEQ ID NO:3 include peptides having from 1 to 5 amino acid substitutions, insertions, or deletions (e.g., 1, 2, 3, 4, or 5 amino acid substitutions, insertions, or deletions collectively) with respect to SEQ ID NO:3, although in some embodiments the Asp at positions 13 and 16 are maintained. In some embodiments, the sequence DINDV is maintained in the derivative. Amino acid substitutions in SEQ ID NO:3 can optionally be at positions occupied by an X at the corresponding position of SEQ ID NO:1. That is, the peptide may have the amino acid sequence of SEQ ID NO:4: LRRFSTXPXXXXDINDVXNF, where X is a standard amino acid or non-genetically encoded amino acid. In some embodiments, X at position 7 is M, A, or G; X at position 9 is F, A, Y, or G; X at position 10 is M, A, G, D-Alanine (dA), or norleucine (Nle); X at position 11 is F, A, Y, G, or 4-chlorophenylalanine (4-ClPhe); X at position 12 and position 18 are independently selected from 2-Aminobutyric acid (Abu), G, S, A, V, T, I, L, or Allylglycine (AllylGly).

In some embodiments, amino acid substitutions are made at any position of a peptide of SEQ ID NO:1, 2, 3, or 4, which can be independently selected from conservative or non-conservative substitutions. In these or other embodiments, the peptide includes from 1 to 10 amino acids added to one or both termini (collectively). The N- and/or C-termini may optionally be occupied by another chemical group (other than amine or carboxy, e.g., amide or thiol), and which can be useful for conjugation of other moieties, including PEG or PLGA-PEG co-polymers, as described in further detail herein.

Conservative substitutions may be made, for instance, on the basis of similarity in polarity, charge, size, solubility, hydrophobicity, hydrophilicity, and/or the amphipathic nature of the amino acid residues involved. The 20 genetically encoded amino acids can be grouped into the following six standard amino acid groups:

(1) hydrophobic: Met, Ala, Val, Leu, Ile;

(2) neutral hydrophilic: Cys, Ser, Thr; Asn, Gln;

(3) acidic: Asp, Glu;

(4) basic: His, Lys, Arg;

(5) residues that influence chain orientation: Gly, Pro; and

(6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. Some preferred conservative substitutions within the above six groups are exchanges within the following sub-groups: (i) Ala, Val, Leu and Ile; (ii) Ser and Thr; (iii) Asn and Gln; (iv) Lys and Arg; and (v) Tyr and Phe.

As used herein, “non-conservative substitutions” are defined as exchanges of an amino acid by another amino acid listed in a different group of the six standard amino acid groups (1) to (6) shown above.

In various embodiments, the biomimetic peptide or peptide agent is a peptide of from about 8 to about 30 amino acids, or from about 10 to about 20 amino acids, and has at least 4, at least 5, or at least 6 contiguous amino acids of SEQ ID NO: 1 or 3. In some embodiments, the peptide contains at least one, at least two, or at least three D-amino acids. In some embodiments, the peptide contains from one to about five (e.g., 1, 2, or 3) non-genetically encoded amino acids, which are optionally selected from 2-Aminobutyric acid (Abu), norleucine (Nle), 4-chlorophenylalanine (4-ClPhe), and Allylglycine (AllylGly).

Exemplary biomimetic peptides in accordance with the disclosure include:

(SEQ ID NO: 5)
LRRFSTMPFMF(Abu)NINNV(Abu)NF,
(SEQ ID NO: 6)
LRRFSTMPAMF(Abu)NINNV(Abu)NF,
(SEQ ID NO: 7)
LRRFSTMPFAF(Abu)NINNV(Abu)NF,
(SEQ ID NO: 8)
LRRFSTMPFMA(Abu)NINNV(Abu)NF,
(SEQ ID NO: 9)
LRRFSTMPF(Nle)F(Abu)NINNV(Abu)NF,
(SEQ ID NO: 10)
LRRFSTMPFM(4-ClPhe)(Abu)NINNV(Abu)NF,
(SEQ ID NO: 11)
LRRFSTMPFMFSNINNVSNF,
(SEQ ID NO: 12)
LRRFSTMPFMFANINNVANF,
(SEQ ID NO: 13)
LRRFSTMPFMFININNVINF,
(SEQ ID NO: 14)
LRRFSTMPFMFTNINNVTNF,
(SEQ ID NO: 15)
LRRFSTMPFMF(AllyGly)NINNV(AllyGly)NF ,
(SEQ ID NO: 16)
LRRFSTMPFMFVNINNVVNF,
(SEQ ID NO: 17)
LRRFSTMPFdAFININNVINF,
(SEQ ID NO: 18)
LRRFSTMPFAFININNVINF,
(SEQ ID NO: 19)
LRRFSTAPFAFININNVINF,
(SEQ ID NO: 20)
LRRFSTAPFdAFIDINDVINF,
(SEQ ID NO: 21)
LRRFSTAPFAFIDINDVINW,
(SEQ ID NO: 22)
dLRRdLRRFSTAPFAFIDINDVINF,
(SEQ ID NO: 23)
LRRFSTAPFAFIDINDVINdF,
(SEQ ID NO: 24)
dLRRFSTAPFAFIDINDVINdF.
(SEQ ID NO: 25)
F(Abu)NINNV(Abu)N,
(SEQ ID NO: 26)
FTNINNVTN,
(SEQ ID NO: 27)
FININNVINF,
(SEQ ID NO: 28)
FSNINNVSNF,
(SEQ ID NO: 29)
FANINNVANF,
(SEQ ID NO: 30)
F(AllyGly)NINNV(AllyGly)NF,
(SEQ ID NO: 31)
FVNINNVVNF,
(SEQ ID NO: 32)
FIDINDVINF,
(SEQ ID NO: 33)
FIDINDVINW,
(SEQ ID NO: 34)
FTDINDVTN,
(SEQ ID NO: 35)
A(Abu)NINNV(Abu)NF,
or
(SEQ ID NO: 36)
(4-ClPhe)(Abu)NINNV(Abu)NF.

The biomimetic peptides or peptide agents can be chemically synthesized and purified using well-known techniques, such as solid-phase synthesis. See U.S. Pat. No. 9,051,349, which is hereby incorporated by reference in its entirety.

Peptides may be provided in the form of a pharmaceutically acceptable salt in some embodiments, or complexed with other components or encapsulated in particles for targeted or sustained delivery to particular tissues.

The biomimetic peptide or peptide agent in some embodiments is formulated as a pharmaceutically acceptable salt. Pharmaceutically acceptable salts are generally well known to those of ordinary skill in the art, and may include, by way of example, but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). Pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

The biomimetic peptide or peptide agent may be formulated for local or systemic delivery, for example, using a variety of pharmaceutically acceptable carriers, including, but not limited to, water, saline, dextrose solutions, human serum albumin, liposomes, hydrogels, microparticles and nanoparticles.

In some embodiments, an effective amount of the biomimetic peptide or peptide agent will be within the range of from about 0.1 to about 50 mg per dose, or in some embodiments, from about 0.5 to about 25 mg per dose, from about 1 to about 10 mg per dose, or from about 1 to about 5 mg per dose, or from about 1 to about 3 mg per dose. The exact dosage will depend upon, for example, the route of administration, the form in which the compound is administered, and the medical condition and/or patient to be treated. In various embodiments, the peptide is administered from 1 to 3 times daily, weekly, or monthly (e.g., once daily, weekly, or monthly), or in some embodiments, no more than once every other month, or no more than once every three months, or no more than once every four months.

In some embodiments, the biomimetic peptide or peptide agent is administered by intravitreal injection, for example, for the treatment of diabetic macular edema, retinal vein occlusion, wet age-related macular degeneration (wet AMD), or diabetic retinopathy. A composition comprising the biomimetic peptide or peptide agent may be administered for the treatment of a condition that is refractory or only partially-responsive to VEGF blockade therapy or kinase inhibitor therapy. For example, the biomimetic peptide may be administered after unsuccessful VEGF blockade therapy, and/or may be administered as the primary, first-line therapy (without other agents). In some embodiments, the peptide is administered at a dose of from about 100 μg to about 1000 μg, or in some embodiments, at a dose of from about 200 μg to about 800 μg, or at a dose of from about 400 to about 800 μg. In some embodiments, the dose of the peptide is about 200 μg, about 400 μg, about 500 μg, about 600 μg, about 800 μg, or about 1 mg. The peptide dose may be administered monthly, every other month, or once every three months, or once every four months, or once every six months. Because the naked peptide can form a depot upon intravitreal injection, the frequency of dosing can be substantially reduced, with or without formulation into particles. Formulation with microparticles can lead to even less frequent dosing, and in some embodiments the formulation comprises both free and encapsulated protein to provide an initial dose of active agent, with a subsequent, sustained release over several months. Even in the absence of microparticle formulation, intravitreal injection at a frequency of about monthly, or every other month, or once every third month is possible. In some embodiments, the peptide formulation comprises microparticles encapsulating a dose of from 1 mg to about 10 mg of peptide agent, or in some embodiments, a dose of from about 1 mg to 5 mg of peptide, or in some embodiments, a dose of from 1 mg to 3 mg of peptide agent.

The biomimetic peptide may be formulated for a variety of modes of administration, including systemic and topical or localized administration. Techniques and formulations generally may be found in Remington: The Science and Practice of Pharmacy (20th ed.) Lippincott, Williams & Wilkins (2000). To aid in bioavailability, the compositions of the disclosure may be delivered in a nano- or micro-particles, or conjugated to polyethylene glycol or other PK-enhancing conjugates, such as fusion with an antibody Fc domain or albumin amino acid sequence. The agents may be delivered, for example, in a timed- or sustained release form. Techniques for formulation and administration may be found in Remington: The Science and Practice of Pharmacy (20th ed.) Lippincott, Williams & Wilkins (2000). Suitable routes may include oral, buccal, by inhalation aerosol, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intratumoral, intracranial, intraperitoneal, intranasal, or intraocular (e.g., intravitreal) injections or other modes of delivery.

For injection, the biomimetic peptides or peptide agents may be formulated and diluted in aqueous solutions, such as in physiologically compatible buffers such as Hank's solution, Ringer's solution, or physiological saline buffer.

For transmucosal administration, penetrants appropriate to the barrier to be permeated are used in the formulation. Such penetrants are generally known in the art.

The compositions can be formulated readily using pharmaceutically acceptable carriers well known in the art into dosages suitable for oral administration. Such carriers enable the biomimetic peptides or peptide agents to be formulated as tablets, pills, capsules, liquids, gels, syrups, slurries, suspensions and the like, for oral ingestion by a subject (e.g., patient) to be treated.

For nasal or inhalation delivery, the biomimetic peptides or peptide agents may be formulated by methods known to those of skill in the art, and may include, for example, but not limited to, examples of solubilizing, diluting, or dispersing substances such as, saline, preservatives, such as benzyl alcohol, absorption promoters, and fluorocarbons.

In some embodiments, the peptide is formulated with a polymeric nanoparticle or microparticle carrier. For example, in some embodiments, the microparticle or nanoparticle comprises a material having one or more degradable linkages, such as an ester linkage, a disulfide linkage, an amide linkage, an anhydride linkage, and a linkage susceptible to enzymatic degradation. In particular embodiments, the microparticle or nanoparticle comprises a biodegradable polymer or a blend of polymers selected from the group consisting of poly(lactic-co-glycolic acid) (PLGA), poly(beta-amino ester) (PBAE), polycaprolactone (PCL), polyglycolic acid (PGA), polylactic acid (PLA), poly(acrylic acid) (PAA), poly-3-hydroxybutyrate (P3HB) and poly(hydroxybutyrate-co-hydroxyvalerate). In some embodiments, the particles comprise a blend of PLGA and PBAE. In other embodiments, nondegradable polymers that are used in the art, such as polystyrene, are blended with a degradable polymer or polymers from above to create a copolymer system. Accordingly, in some embodiments, a nondegradable polymer is blended with the biodegradable polymer.

In some embodiments, the invention provides a nanoparticle comprising PLGA-PEG copolymers and a conjugated biomimetic peptide. The conjugated peptide can be a peptide of any one of SEQ ID NOs:1-36.

In some embodiments, the nanoparticles contain an additional drug or targeting agent conjugated to the surface of the nanoparticle. For example, the nanoparticles may be made from PLGA-PEG-X and PLGA-PEG-Y polymers, where X is the biomimetic peptide and Y is another drug or targeting agent. The targeting agent may be a tissue selective targeting agent, or may be selective for a desired cell type, including cancer cells. Nanoparticles in these embodiments (having conjugated peptide, and optionally an additional targeting agent) may be used in a treatment of cancer, including solid tumors as described above, and including glioblastoma or breast cancer (including triple-negative breast cancer).

Other target binding agents may be used, in addition or alternatively (including alternative integrin-binding moieties), and these include antibodies and antigen-binding portions thereof. The various formats for target binding include a single-domain antibody, a recombinant heavy-chain-only antibody (VHH), a single-chain antibody (scFv), a shark heavy-chain-only antibody (VNAR), a microprotein (cysteine knot protein, knottin), a DARPin, a Tetranectin, an Affibody; a Transbody, an Anticalin, an AdNectin, an Affilin, a Microbody, a peptide aptamer, a phylomer, a stradobody, a maxibody, an evibody, a fynomer, an armadillo repeat protein, a Kunitz domain, an avimer, an atrimer, a probody, an immunobody, a triomab, a troybody, a pepbody, a vaccibody, a UniBody, a DuoBody, a Fv, a Fab, a Fab′, a F(ab′)2, a peptide mimetic molecule, or a synthetic molecule, or as described in US Patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In some embodiments, the nanoparticle is synthesized from poly(lactic-co-glycolic acid) polyethylene glycol (PLGA-PEG) block copolymers of tunable size which are covalently linked to the peptide of any one of SEQ ID NOs:1-36, or derivative thereof, or other binding agent as described above. A mix of conjugated and unconjugated polymers in any ratio can be used to create nanoparticles with the desired density of targeting agent on the surface. In some embodiments, the biomimetic peptide comprises the amino acid sequence of SEQ ID NO:3 (referred to as AXT107 or as SP2043).

In some embodiments, the peptide that is conjugated to the particle has the amino acid sequence of SEQ ID NOs:1-36, or derivative thereof. The nanoparticles in some embodiments are formed from PLGA-PEG-peptide conjugates, or in other embodiments, the peptide is conjugated to pre-formed particles.

As used herein, the term “nanoparticle,” refers to a particle having at least one dimension in the range of about 1 nm to about 1000 nm, including any integer value between 1 nm and 1000 nm (including about 1, 2, 5, 10, 20, 50, 60, 70, 80, 90, 100, 200, 500, and 1000 nm and all integers and fractional integers in between). In some embodiments, the nanoparticle has at least one dimension, e.g., a diameter, of about 50 to about 100 nm. In some embodiments, the nanoparticle has a diameter of about 70 to 100 nm.

In some embodiments, the particle is a microparticle. The term “microparticle” includes particles having at least one dimension in the range of at least about one micrometer (μm). The term “particle” as used herein is meant to include nanoparticles and microparticles.

The particles may be designed to provide desired pharmacodynamic advantages, including circulating properties, biodistribution, and degradation kinetics. Such parameters include size, surface charge, polymer composition, ligand conjugation chemistry, and peptide conjugation density, among others. For example, in some embodiments, the particles have a PLGA polymer core, and a hydrophilic shell formed by the PEG portion of PLGA-PEG co-polymers, wherein a portion of the PLGA-PEG polymers have a terminal attachment of the peptide. The hydrophilic shell may further comprise ester-endcapped PLGA-PEG polymers that are inert with respect to functional groups, such as PLGA-PEG-MeOH polymers. In some embodiments, some or all of the unconjugated polymers have other terminal groups (such as carboxy) to provide fine tuning of the surface properties.

Peptides described herein can be chemically conjugated to the particles using any available process. Functional groups for peptide conjugation include PEG-COOH, PEG-NH2, PEG-SH. See, e.g., Hermanson, BIOCONJUGATE TECHNIQUES, Academic Press, New York, 1996. Activating functional groups include alkyl and acyl halides, amines, sulfhydryls, aldehydes, unsaturated bonds, hydrazides, isocyanates, isothiocyanates, ketones, and other groups known to activate for chemical bonding. Alternatively, peptides can be conjugated through the use of a small molecule-coupling reagent. Non-limiting examples of coupling reagents include carbodiimides, maleimides, N-hydroxysuccinimide esters, bischloroethylamines, bifunctional aldehydes such as glutaraldehyde, anhydrides and the like.

In an exemplary embodiment, the nanoparticles have a core (PLGA) that can be tuned for a specific biodegradation rate in vivo (by adjusting the LA:GA ratio and/or molecular weight of the PLGA polymer). In some embodiments, the PLGA is based on a LA:GA ratio of from 20:1 to 1:20, including compositions of L/G of: 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. PLGA degrades by hydrolysis of its ester linkages. The time required for degradation of PLGA is related to the ratio of monomers:the higher the content of glycolide units, the lower the time required for degradation as compared to predominantly lactide units. In addition, polymers that are end-capped with esters (as opposed to the free carboxylic acid) have longer degradation half-lives.

In some embodiments, the PLGA polymers have a molecular weight in the range of about 10K to about 70K, such as about 20K, about 25K, about 30K, about 40K, about 50K, about 60K, or about 70K, to provide tunable particle size. The PEG portion of the polymer is generally in the range of 2K to 5K. In various embodiments, the ratio of PLGA-PEG-peptide and unconjugated PLGA-PEG ranges from about 1:20 to about 20:1, such as from about 1:15 to about 15:1, or about 1:10 to about 10:1, or about 1:5 to about 5:1, or about 1:2 to about 2:1. In some embodiments, the ratio of PLGA-PEG-peptide and unconjugated copolymers is about 1:1. In some embodiments, at least 50% of the polymers have conjugated peptide. In some embodiments, the nanoparticle has a size (average diameter) within the range of about 50 to about 200 nm, or within the range of about 50 to about 100 nm. In some embodiments, the nanoparticle has a zeta potential in deionized water within the range of about −5 mV to about −40 mV, and in some embodiments, from about −10 mV to about −30 mV (e.g., about −20, about −25, or about −30 mV).

In some embodiments, the nanoparticle further comprises an encapsulated active agent, which may be an active agent disclosed herein for treatment of a Tie2-related condition, such as a condition characterized by microvascular or lymphatic leakage, including flu, Alzheimer's Disease, hemorrhagic fever, cerebral malaria, tumor growth or metastasis, and others described herein. In these embodiments, the nanoparticle provides a sustained release of the active agent. For example, in some embodiments, the active agent is a chemotherapeutic agent, such as one or more of: aminoglutethimide, amsacrine, anastrozole, asparaginase, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

While the nanoparticle is substantially spherical in some embodiments, the nanoparticle may optionally be non-spherical.

There are various physical and chemical properties that can affect how a material interacts with a biological system. In the case of microparticle- and nanoparticle-based materials, the choice of material, the size distribution, and the shape distribution of the particles are all critical parameters affecting the particles' activity. It has been previously shown that both the size and shape of a particle can affect the way the particle interacts with various cells of the body. For example, the shape of the particle can affect how well various cell types can uptake the particle, where an ellipsoidal particle is usually more difficult for a cell to uptake than a spherical particle. Stretching the shape of the particles can therefore reduce unwanted uptake of particles, such as by the immune system cells, thereby extending the half-life of the particles in the body. The particle sizes also affect the ability of cells to uptake and interact with the particles. Optimization of the activity of a particle based system can therefore be achieved by tuning the size and shape distribution of the particles.

In some embodiments, the dimensions of the nanoparticle and/or process for stretching the particles as disclosed in WO 2013/086500, which is hereby incorporated by reference in its entirety.

In particular embodiments, the three-dimensional microparticle or nanoparticle comprises a prolate ellipsoid, wherein the dimension (a) along the x-axis is greater than the dimension (b) along the y-axis, and wherein the dimension (b) along the y-axis is substantially equal to the dimension (c) along the z-axis, such that the prolate ellipsoid can be described by the equation a>b=c. In other embodiments, the ellipsoid is a tri-axial ellipsoid, wherein the dimension (a) along the x-axis is greater than the dimension (b) along the y-axis, and wherein the dimension (b) along the y-axis is greater than the dimension (c) along the z-axis, such that the tri-axial ellipsoid can be described by the equation a>b>c. In yet other embodiments, the ellipsoid is an oblate ellipsoid, wherein the dimension (a) along the x-axis is equal to the dimension (b) along the y-axis, and wherein the dimension (b) along the y-axis is greater than the dimension (c) along the z-axis, such that the oblate ellipsoid can be described by the equation a=b>c. The presently disclosed asymmetrical particles, however, do not include embodiments in which a=b=c.

In still other embodiments, the microparticle or nanoparticle has an aspect ratio ranging from about 1.1 to about 5. In other embodiments, the aspect ratio has a range from about 5 to about 10. In some embodiments, the aspect ratio has a range from about 1.5 to about 3.5.

In some embodiments, the particle is a microparticle that encapsulates a drug cargo (such as a peptide described herein, and/or another agent). In these embodiments, the particle may or may not contain peptide conjugated to its surface. In these embodiments, the particle can provide a long acting drug depot, to provide a sustained release of peptide. Exemplary particle formats include those described in WO 2014/197892, which is hereby incorporated by reference. In some embodiments, particles do not incorporate poly(beta-amino ester) (PBAE), and thus the polymers consist essentially of PLGA-PEG block co-polymers. These particles can be used for intraocular injection, for example, as a treatment for macular degeneration (e.g., wet or dry age-related macular degeneration) or diabetic macular edema. In some embodiments, the cargo allows for a combination of active agents to be delivered to desired site. In some embodiments, the nanoparticle is administered for the treatment of cancer. In these or other embodiments, the particle has a size (average diameter) in the range of 1 μm to 500 μm, such as in the range of about 1 μm to about 250 μm. The particles can be injected from about once daily to about once every six months, or about weekly or about monthly, depending on the duration of the sustained peptide or drug release.

In certain aspects, the invention provides a method for preventing or treating a condition involving Tie-2-related vascular permeability or lymphatic vessel integrity in a patient. The method comprises administering the collagen IV-derived biomimetic peptide, or nanoparticle formulation thereof, to the patient in an amount effective to reduce Tie-2-dependent vascular or lymphatic permeability. Restoration of Tie2 activation provides therapeutic benefit in conditions associated with edema or vascular permeability, including macular edema, diabetic macular edema (DME), and other conditions, including conditions characterized by acute or chronic inflammation. Tie2-related conditions include diabetic macular edema, retinal vein occlusion, wet AMD, background diabetic retinopathy, cancer (including for reducing, slowing or preventing tumor growth or metastasis), influenza, hemorrhagic fever, cerebral malaria, Alzheimer's disease, acute respiratory distress syndrome, pulmonary edema, asthma, Respiratory Syncytial Virus, SARS, pneumonia, sepsis among others.

In various embodiments, the biomimetic peptide can be delivered for conditions (including macular edema, wet AMD, tumor growth or metastasis) that are refractory or only partially-responsive to vascular endothelial growth factor (VEGF) blockade or inhibitor therapy. Pharmaceutical agents that block VEGF include aflibercept, bevacizumab, ranibizumab, and ramucirumab, and similar agents, which are administered to slow or block angiogenesis. Other agents that target VEGF-mediated biological activity include kinase inhibitors such as pazopanib, sorafenib, sunitinib, axitinib, ponatinib, lenvatinib, vandetanib, regorafenib, and cabozantinib.

Aflibercept is a biopharmaceutical drug for the treatment of wet macular degeneration (EYLEA), and for metastatic colorectal cancer as (ZALTRAP). Aflibercept is an inhibitor of VEGF, and is a recombinant fusion protein consisting of vascular endothelial growth factor (VEGF)-binding portions from the extracellular domains of human VEGF receptors 1 and 2, that are fused to the Fc portion of the human IgG1 immunoglobulin. Aflibercept binds to circulating VEGFs and acts like a “VEGF trap”, inhibiting the activity of the vascular endothelial growth factor subtypes VEGF-A and VEGF-B, as well as to placental growth factor (PGF), inhibiting the growth of new blood vessels in the choriocapillaris or the tumor, respectively.

Bevacizumab (AVASTIN) is an angiogenesis inhibitor, a drug that slows the growth of new blood vessels. Bevacizumab is a recombinant humanized monoclonal antibody that blocks angiogenesis by inhibiting VEGF-A. Bevacizumab is administered for treating certain metastatic cancers, including colon cancer, lung cancers (e.g., NSCLC), renal cancers, ovarian cancers, breast cancer, and glioblastoma. Bevacizumab can also be used for treatment of eye diseases, including AMD and diabetic retinopathy.

Ranibizumab (LUCENTIS) is a monoclonal antibody fragment (Fab), and is administered for treatment of wet AMD. The drug is injected intravitreally (into the vitreous humour of the eye) about once a month. Ranibizumab is a monoclonal antibody that inhibits angiogenesis by inhibiting VEGF A, similar to Bevacizumab.

Thus, in some embodiments, the VEGF inhibitor comprises a monoclonal antibody or antigen-binding portion thereof, or comprises extracellular domains of human VEGF receptors 1 and/or 2. For example, the biomimetic peptide may be administered after unsuccessful VEGF blockade therapy, that is, where reductions in angiogenesis, lymphangiogenesis, and/or edema were not observed. In some embodiments, the peptide is administered as an alternative to VEGF blockade therapy. In still further embodiments, the peptide is administered in combination with VEGF blockade therapy, either simultaneously with, before, or after a VEGF blockade regimen. By activating Tie2 signaling, the biomimetic peptides or peptide agents provide therapeutic benefits that may not be observed with VEGF blockage therapy, or VEGF blockade therapy alone.

In some embodiments, the patient has macular edema. Macular edema occurs when fluid and protein deposits collect on or under the macula of the eye (a yellow central area of the retina) and causes it to thicken and swell. The causes of macular edema include chronic or uncontrolled diabetes type 2 (e.g., diabetic retinopathy), in which peripheral blood vessels including those of the retina leak fluid into the retina. Other causes and/or associated disorders include age-related macular degeneration (AMD), chronic uveitis, atherosclerosis, high blood pressure and glaucoma. In some embodiments, the patient has or is at risk of retinal vein occlusion, which can lead to severe damage to the retina and blindness, due to ischemia and edema. In some embodiments, the patient receives intra-ocular injection of the peptide or particle formulation thereof, in combination with or as an alternative to VEGF blockade therapy.

In some embodiments, the patient has or is at risk of flu. Influenza (“the flu”) is an infectious disease caused by the influenza virus. Symptoms include a high fever, runny nose, sore throat, muscle pains, headache, coughing, and fatigue. These symptoms typically begin two days after exposure to the virus. The infection may be confirmed by testing the throat, sputum, or nose for the presence of the virus. Antiviral drugs, such as the neuraminidase inhibitors (e.g., oseltamivir, among others) have been used to treat influenza, and while they have shown modest benefits, they must be used early in the infection (e.g., soon after symptoms appear) to provide benefit. Approximately 33% of people with influenza are asymptomatic. Symptoms of influenza can start quite suddenly around one to two days after infection. Usually the first symptoms are chills or a chilly sensation, but fever is also common early in the infection. Anti-viral treatments, although sometimes providing modest benefits, run the risk of viral resistance, which would be particularly problematic in a potent pandemic strain.

An attractive alternative to treating the virus is to treat the host response, which is much less likely to result in resistance to the drug, and may provide a greater window of efficacy in allowing treatment of more advanced stages of the illness. One of the major responses by the host is an inflammatory response that causes pulmonary microvascular leak and lung injury sometimes leading to respiratory failure. Anti-edemic agents that inhibit microvascular leak could ameliorate the symptoms of the flu.

In some embodiments, the peptide or pharmaceutical composition comprising the same is first administered before the appearance of flu symptoms. For example, the patient may be diagnosed as having flu using a laboratory test that detects the presence of the virus in patient samples, or the patient is at risk of flu after being exposed to the virus. Exposure can be determined by close contact with infected and/or symptomatic individuals.

In other embodiments, the peptide or pharmaceutical composition is first administered after the first flu symptoms appear. In some embodiments, the peptide or pharmaceutical composition is administered within 1 to 4 days (such as 1 or 2 days) after the appearance of the first flu symptoms. In accordance with this aspect of the invention, the peptide reduces edema in the lung associated with influenza virus, thereby ameliorating the symptoms and/or severity of the condition. In some embodiments, the overall length of the illness can be reduced by one, two, three, four, or more days, and/or the severity and discomfort can be substantially reduced.

For treatment of a patient having or at risk of flu, the peptide or pharmaceutical composition described herein can be administered from about 1 to about 5 times daily, such as from about 1 to about 3 times daily. In some embodiments, the peptide is administered locally to the lungs, for example, by powder or solution aerosol, or in other embodiments is administered systemically.

In some embodiments, the peptide is administered with one or more anti-viral agents that are active against influenza, or alternatively is administered with one or more anti-inflammatory agents, either as a separate drug formulations or as a co-formulated product. Exemplary anti-viral agents include Tamiflu® (oseltamivir phosphate), Relenza® (zanamivir), Rapivab (peramivir), amantadine, and rimantadine. Anti-inflammatory agents include NSAIDs such as aspirin, ibuprofen, acetaminophen, and naproxen.

In other embodiments, the peptide or pharmaceutical composition is administered for the treatment of, or to slow the progression of, Alzheimer's disease. The blood-brain barrier (BBB) limits entry of blood-derived products, pathogens, and cells into the brain that is essential for normal neuronal functioning and information processing. Post-mortem tissue analysis indicates BBB damage in Alzheimer's disease. The timing of BBB breakdown remains, however, elusive. Advanced dynamic contrast-enhanced MRI with high spatial and temporal resolutions to quantify regional BBB permeability in the living human brain have shown an age-dependent BBB breakdown in the hippocampus, a region critical for learning and memory that is affected early in Alzheimer's disease. These data suggest that BBB breakdown is an early event in the aging human brain that begins in the hippocampus and may contribute to cognitive impairment. Thus, an agent that inhibits blood-brain damage and the resulting increased permeability could slow down the progress of Alzheimer's disease. Administration of the peptide or compositions described herein in some embodiments, maintain the integrity of the blood-brain barrier, to thereby slow or prevent the onset or progression of Alzheimer's disease.

In some embodiments, the patient is undergoing treatment with at least one additional agent for treatment of Alzheimer's disease, which may be selected from acetylcholinesterase inhibitors (tacrine, rivastigmine, galantamine and donepezil) or memantine.

For treatment of a patient showing potential symptoms of Alzheimer's disease, particularly early stage disease, the peptide or pharmaceutical composition described herein can be administered from about 1 to about 5 times daily, such as from about 1 to about 3 times daily to slow the onset or progression of the disease. Early stage disease can often be observed as an increasing impairment of learning and memory, which eventually leads to a definitive diagnosis. In some, difficulties with language, executive functions, perception (agnosia), or execution of movements (apraxia) are more prominent than memory problems. Language problems are characterized by a shrinking vocabulary and decreased word fluency, leading to a general impoverishment of oral and written language.

In other embodiments, the patient has or is at risk of a hemorrhagic fever or syndrome, which are caused by hemorrhagic viruses. The most notorious of these are the Ebola and the Marburg viruses. Bleeding also occurs in people with Dengue or Lassa fever. In Ebola this hemorrhagic syndrome occurs somewhat late in the disease, typically 24 to 48 hours before death. Cases with bleeding can be dramatic and may occur from the nose, mouth and other orifices of the body. The mechanisms leading to the bleeding are known in broad outline: the virus causes up-regulation of clotting factors which are produced by the liver, the increased number of clotting factors cause clots to form in small blood vessels, the supply of clotting factors produced by the liver is exhausted because the liver is under attack by the virus, the hyper-activated immune system increases production of inflammatory proteins that cause the blood vessels to start bleeding, the unavailability of clotting factors means that the bleeding cannot be stemmed. Many deaths occur even without bleeding but patients with bleeding have a very high mortality rate. Agents administered after symptoms first appear could stop or reduce bleeding from the microvasculature in patients who would otherwise progress to display hemorrhagic syndrome.

In some embodiments, the patient has Ebola virus or Marburg virus. For example, the patient may have early signs of hemorrhagic fever, such as fever and increased susceptibility to bleeding, and/or flushing of the face and chest, small red or purple spots (petechiae). Other signs and symptoms of hemorrhagic fever include malaise, muscle pain, headache, vomiting, and diarrhea. In some embodiments, the presence of Ebola virus or other hemorrhagic fever virus is confirmed in patient samples. In some embodiments, the patient is undergoing treatment with at least one anti-viral agent or anti-inflammatory or agent for treatment of the hemorrhagic fever, such as intravenous ribavirin. For treatment of a patient having or at risk of hemorrhagic fever, the peptide or pharmaceutical composition described herein can be administered from about 1 to about 5 times daily, such as from about 1 to about 3 times daily, to slow the progression of the disease.

In still other embodiments, the patient has or is at risk of cerebral malaria (CM). CM is one of the most lethal complications of Plasmodium falciparum malaria and accounts for a large fraction of the malaria-related deaths. The World Health Organization (WHO) defines CM as coma (incapacity to localize a painful stimulus or Blantyre coma score ≤2) persisting at least 1 hour after termination of a seizure or correction for hypo-glycemia in the presence of asexual P. falciparum parasitemia and without the presence of other causes of encephalopathy. Up to 75% of CM-related deaths occur within 24 hours of admission. Multimodal magnetic resonance techniques such as imaging, diffusion, perfusion, angiography, spectroscopy have shown that vascular damage including blood-brain barrier disruption and hemorrhages occur in CM. These effects are thought to be due to inflammatory processes. Penet et al., (J Neurosci. 2005 Aug. 10; 25(32):7352-8) have shown using a mouse model of CM that major edema formation as well as reduced brain perfusion occurs in CM and is accompanied by an ischemic metabolic profile with reduction of high-energy phosphates and elevated brain lactate. They also used angiography which provided compelling evidence for major hemodynamics dysfunction. Importantly they found that edema further worsens ischemia by compressing cerebral arteries subsequently leading to a collapse of the blood flow that ultimately is the cause of death. These findings demonstrate the coexistence of inflammatory and ischemic lesions and prove the major role of edema in the fatal outcome of experimental cerebral malaria. Agents that inhibit edema and/or ischemia in the brain could be used in combination with anti-malarial agents that directly target the parasite to improve treatment of these patients. In some embodiments, the patient receives an anti-malarial therapy selected from chloroquine, mefloquine, doxycycline, or the combination of atovaquone and proguanil hydrochloride (Malarone).

In these embodiments, the peptide maintains the blood brain barrier and vascular integrity in patients with cerebral malaria. For treatment of a patient having or at risk of cerebral malaria, the peptide or pharmaceutical composition described herein can be administered from about 1 to about 5 times daily, such as from about 1 to about 3 times daily, to slow the progression of the disease and/or prevent death.

In other aspects, the invention provides a method for treating cancer, including normalizing the tumor vasculature for chemotherapy, or preventing or slowing tumor growth or metastasis. Angiogenesis has been widely viewed as a drug target for treating cancer. VEGF and its receptor VEGFR2 are important mediators of angiogenesis. Bevacizumab, an antibody that sequesters human VEGF, as well as Aflibercept and Ranibizumab, and small molecule tyrosine kinase inhibitors that inhibit VEGFR2, have been administered as treatments for various types of cancer. In addition to its well-known pro-angiogenic activity, VEGF also functions as an immune suppressor by inhibiting the maturation of dendritic cells. Tumors are thought to produce VEGF both to attract neovasculature and to suppress the immune system by reducing the number of mature immune cells and modulating lymphocyte endothelial trafficking. In some embodiments, the cancer is non-responsive to such agents (e.g., after treatment with one or more of such agents), including aflibercept, bevacizumab, ranibizumab, ramucirumab, pazopanib, sorafenib, sunitinib, axitinib, ponatinib, lenvatinib, vandetanib, regorafenib, and cabozantinib.

In some embodiments the cancer is a sarcoma, carcinoma, or solid tumor cancer selected from germ line tumors, tumors of the central nervous system, breast cancer, prostate cancer, cervical cancer, uterine cancer, lung cancer, ovarian cancer, testicular cancer, thyroid cancer, astrocytoma, glioma, pancreatic cancer, stomach cancer, liver cancer, colon cancer, melanoma (including advanced melanoma), renal cancer, bladder cancer, esophageal cancer, cancer of the larynx, cancer of the parotid, cancer of the biliary tract, rectal cancer, endometrial cancer, squamous cell carcinomas, adenocarcinomas, small cell carcinomas, neuroblastomas, mesotheliomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, lymphomas, melanomas, non-rhabdomyosarcome soft tissue sarcomas, osteosarcomas, peripheral primative neuroectodermal tumors, retinoblastomas, rhabdomyosarcomas, and Wilms tumors. In some embodiments, the cancer is non-small cell lung cancer, melanoma, prostate cancer, metastatic renal cell cancer.

In some embodiments, the cancer is triple-negative breast cancer (TNBC), small cell lung cancer (SCLC), glioblastoma, or liver cancer.

In various embodiments, the patient can have either early stage cancer (e.g., stage I or II), or be in later stages (stage III or stage IV). Stage I cancers are localized to one part of the body. Stage II cancers are locally advanced, as are Stage III cancers. Whether a cancer is designated as Stage II or Stage III can depend on the specific type of cancer. For example, stage II can indicate affected lymph nodes on only one side of the diaphragm, whereas stage III indicates affected lymph nodes above and below the diaphragm. The specific criteria for stages II and III therefore differ according to diagnosis. Stage IV cancers have often metastasized, or spread to other organs or throughout the body. The peptide or particle formulation thereof can be administered to prevent progression of Stage I or II cancer, or to slow progression or inhibit further progression of Stage III or Stage IV cancers.

In some embodiments, the cancer is non-resectable, such as non-resectable liver cancer. A non-resectable cancer is a malignancy which cannot be surgically removed, due either to the number of metastatic foci, or because it is in a surgical danger zone.

In some embodiments, the condition is vascular permeability prior to chemotherapy for cancer. For example, a regimen of the biomimetic peptide or peptide agent (e.g., from one to ten doses) may be administered at least one week or at least two weeks prior to receiving cancer chemotherapy, to normalize the tumor vasculature and/or the tumor microenvironment. Exemplary chemotherapeutic agents include aminoglutethimide, amsacrine, anastrozole, asparaginase, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine, among others. In some embodiments, the biomimetic peptide or peptide agent is administered by parental administration, including intratumorally in some embodiments.

In some embodiments, the patient has an inflammatory condition involving lymphatic dysfunction, including lymphangitis (an inflammation of the lymph vessels) and lymphedema (a chronic pooling of lymph fluid in the tissue, which can be a side-effect of some surgical procedures). The lymphatic system performs three major functions in the body: drainage of excess interstitial fluid and proteins back to the systemic circulation; regulation of immune responses by both cellular and humoral mechanisms; and absorption of lipids from the intestine. Lymphatic disorders are seen following malignancy, congenital malformations, thoracic and abdominal surgery, trauma, and infectious diseases. Many lymphatic disorders are encountered in the operating theatre and critical care settings. Administration of the peptide can help restore, or prevent continued decline of, lymphatic vessel integrity.

In some embodiments, the condition is capillary leak syndrome. Systemic capillary leak syndrome is a condition in which fluid and proteins leak out of capillary vessels and flow into surrounding tissues, resulting in dangerously low blood pressure. Attacks frequently last for several days and require emergency care.

In some embodiments, the condition is sepsis. Sepsis is a life-threatening condition that arises when the body's response to infection injures its own tissues and organs. Sepsis is caused by an immune response triggered by an infection. The infection is most commonly bacterial, but it can be from fungi, viruses, or parasites. Common locations for the primary infection include lungs, brain, urinary tract, skin, and abdominal organs. Sepsis is usually treated with intravenous fluids and antibiotics. Disease severity partly determines the outcome, with a high risk of death. Administration of the peptide can help restore, or prevent continued decline of, vascular integrity to ameliorate the condition.

In some embodiments, the condition involves acute or chronic lung inflammation, such as acute respiratory distress syndrome (ARDS), Acute Lung Injury (ALI), chronic asthma, or chronic obstructive pulmonary disorder (COPD). In such embodiments, the peptide composition may be administered locally by inhalation or administered systemically.

Acute respiratory distress syndrome (ARDS) is characterized by widespread inflammation in the lungs, and may be triggered by pathologies such as trauma, pneumonia and sepsis. ARDS is a form of pulmonary edema provoked by an acute injury to the lungs that result in flooding of the microscopic air sacs responsible for the exchange of gases with capillaries in the lungs. In ARDS, these changes are not due to heart failure. The clinical syndrome is associated with pathological findings including pneumonia, eosinophilic pneumonia, cryptogenic organizing pneumonia, acute fibrinous organizing pneumonia, and diffuse alveolar damage (DAD). Of these, the pathology most commonly associated with ARDS is DAD, which is characterized by a diffuse inflammation of lung tissue. The triggering insult to the tissue usually results in an initial release of chemical signals and other inflammatory mediators secreted by local epithelial and endothelial cells. Inflammation, such as that caused by sepsis, causes endothelial dysfunction, fluid leakage from the capillaries and impaired drainage of fluid from the lungs. Elevated inspired oxygen concentration often becomes necessary at this stage, and may facilitate a ‘respiratory burst’ in immune cells. In a secondary phase, endothelial dysfunction causes cells and inflammatory exudate to enter the alveoli. This pulmonary edema increases the thickness of the alveolocapillary space, increasing the distance the oxygen must diffuse to reach the blood, which impairs gas exchange leading to hypoxia, increases the work of breathing and eventually induces fibrosis of the airspace.

In some embodiments, the patient has non-cardiogenic pulmonary edema, which is optionally associated with asthma or chronic obstructive pulmonary disorder (COPD).

In some embodiments the condition is angioedema or urticaria. Angioedema is the rapid swelling of the dermis, subcutaneous tissue, mucosa and submucosal tissues. Urticaria, commonly known as hives, occurs in the upper dermis. Cases where angioedema progresses rapidly are a medical emergency, as airway obstruction and suffocation can occur. In some embodiments, administration of the peptide may reduce the severity of the symptoms.

In some embodiments, the patient has vascular leak syndrome, which is optionally side effect of immunotherapy. Capillary leak syndrome is characterized by self-reversing episodes during which the endothelial cells which line the capillaries are thought to separate for a few days, allowing for a leakage of fluid from the circulatory system to the interstitial space, resulting in a dangerous hypotension (low blood pressure), hemoconcentration, and hypoalbuminemia.

In certain aspects of the disclosure, the invention provides a peptide composition of formulation, including particle formulations. The peptide may have an amino acid sequence of any one of SEQ ID NOs:1-36, including a derivative peptide having a sequence selected from SEQ ID NOs: 5 to 36. In some embodiments, the formulation comprises from 100 μg to about 1000 μg of peptide agent per unit dose, and which optionally does not involve encapsulation into particles. In some embodiments, the formulation comprises from about 1 mg to about 10 mg per unit dose (or in some embodiments from 1 to 5 mg or from 1 to 3 mg), and which may comprise particle encapsulation, optionally with free peptide. Formulations providing both encapsulated and free peptide can provide for an initial dose (e.g., within the range of 100 μg to about 1000 μg), while encapsulated peptide provides a sustained release over several months (e.g., from 3 to 6 months, or more). In some embodiments, the peptide agent has the sequence of SEQ ID NOs: 1, 2, 3, or 4.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example, within plus or minus 10%.

The invention will further be described in accordance with the following non-limiting examples.

### Examples

Regulatory functions of integrins on Ang-Tie signaling are described in the below examples using an exemplary integrin-binding, biomimetic peptide, AXT107. AXT107 is a twenty-mer peptide, derived from a sequence in type IV collagen. AXT107 binds tightly to integrin α₅β₁ and to integrin αᵥβ₃ and disrupts activities of, at least, the growth factor receptors VEGFR2, cMet, PDGFRβ, and IGF1R (Lee et al., Sci Rep. 2014; 4:7139).

As described herein, AXT107 was found to inhibit vascular leakage by a novel mechanism involving Ang2 and Tie2. AXT107 strongly promotes the agonist activity of Ang2 leading to increased phosphorylation of Tie2, Akt and Stat3 in endothelial cells to strengthen the barrier between endothelial cells in the vasculature. AXT107 disrupts interactions between IGF1R and β1 integrin and enhances VEGFR2 degradation in vitro and inhibits the growth and permeability of neovasculature in vivo.

The following examples demonstrate that treatment with the exemplary integrin-binding, biomimetic peptide potentiates the normally weak agonistic activity of Ang2 towards Tie2 both in vitro and in vivo and specifically activates downstream targets associated with EC survival and barrier function. Mechanistically, AXT107 treatment dissociates α₅ integrin and β₁ integrin, resulting in the translocation and activation of Tie2 at EC-EC junctions and decreased monolayer permeability through the reorganization of F-actin and VE-cadherin.

### Example 1: AXT107 Strongly Promotes Agonist Activity of Ang2

The Tie2 receptor tyrosine kinase signaling pathway and its ligands Angiopoietin1 (Ang1) and Angiopoietin2 (Ang2) regulate vascular permeability, which is compromised in patients with macular edema including patients with retinal vein occlusion (RVO), diabetic macular edema (DME), wet age-related macular degeneration (wet AMD), and background diabetic retinopathy (DR). Ang1 binds Tie2 and stimulates phosphorylation and downstream signaling stabilizing blood vessels (1,2). Ang2 competes with Ang1 for Tie2 binding reducing the phosphorylation of Tie2, and thus it acts as an endogenous Tie2 antagonist (3). Ischemic or hypoxic retina produces high levels of Ang2 (4), and Ang2 levels, like that of VEGF levels, are increased in the eyes of DME patients (5). Ang2 increases the responsiveness of retinal vessels to VEGF and promotes vascular leakage and neovascularization (6-9). These results suggest that restoration of Tie2 activation could provide benefit in conditions associated with edema, including macular edema, DME, and others.

Tie2 may also regulate lymphatic vessel integrity especially during inflammation. Specifically, molecules that enhance phosphorylation of Tie2 could potentially be used to treat lymphatic dysfunction during inflammation (10).

Other data suggest that Ang2 also acts as a weak agonist of Tie2 especially when Ang1 levels are low (11). Exogenous Ang2 activates Tie2 and the promigratory, prosurvival PI3K/Akt pathway in endothelial cells (ECs) but with less potency and lower affinity than exogenous Ang1. ECs produce Ang2 but not Ang1. This endogenous Ang2 maintains Tie2, phosphatidylinositol 3-kinase, and Akt activities, and it promotes EC survival, migration, and tube formation.

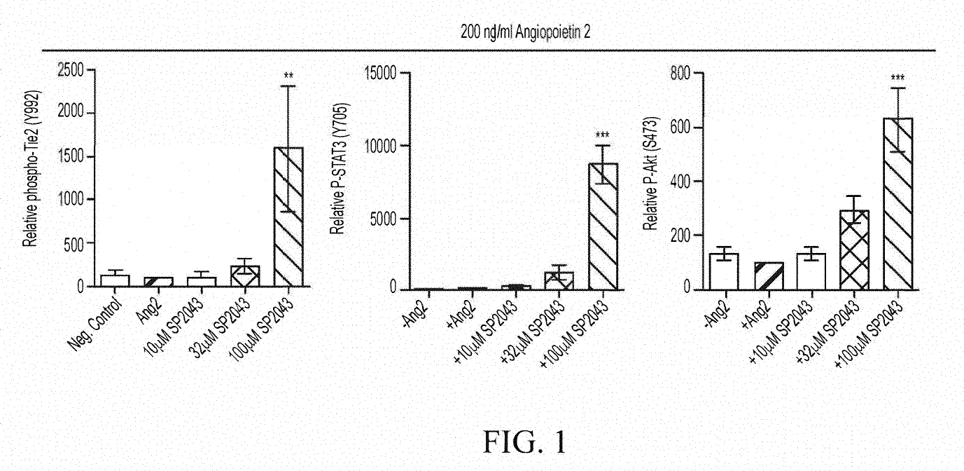

AXT107 is an integrin-binding antiangiogenic biomimetic peptide which inhibits signaling from multiple proangiogenic pathways including VEGF, PDGF, HGF, and IGF1; it represents a class of collagen IV-derived biomimetic peptides. Inhibition of these pathways inhibits neovascularization, and inhibition of the VEGF pathway, in particular, inhibits vascular leakage. As described herein, AXT107 was found to inhibit vascular leakage by a novel mechanism involving Ang2 and Tie2. Normally, both VEGF levels and Ang2 levels are increased in patients with DME and they coordinately promote neovascularization and vascular permeability. As shown in FIG. 1, AXT107 (identified in the figure as SP2043) strongly promotes the agonist activity of Ang2 leading to increased phosphorylation of Tie2, Akt and Stat3 in endothelial cells to strengthen the barrier between endothelial cells in the vasculature.

A confluent monolayer of human microvascular endothelial cells (HMEC) was serum starved with EBM-2 overnight. After a 90 min treatment with AXT107 at concentrations of 0, 10, 32, and 100 μM, the cells were treated for 15 min with 1 mM sodium orthovanadate. Next the cells were treated with Angiopoietin-2 (Ang-2) at 200 ng/mL for 15 min. Following cell lysis, the lysates were immunoblotted for pTie2 (Y992), pSTAT3 (Y705), and pAkt (S473).

This result suggests that AXT107 and other peptides from this class could inhibit vascular leakage in patients with DME and other forms of macular edema by simultaneously inhibiting VEGF and other proangiogenic growth factors and by promoting the phosphorylation of Tie2 by increasing the potency of Ang2 as an agonist. via EFS: Mar. 26,2019 ASX-002/114293-5002

This also applies to other diseases where vascular permeability may be important such as cancer, influenza, hemorrhagic fevers, cerebral malaria and others in which edema is a major contributing factor by enhancing the activity of Tie2 signaling.

### REFERENCES

1. Suri C, Jones P F, Patan S, et al., Requisite role of angiopoietin-1, a ligand for the TIE2 receptor, during embryonic angiogenesis. Cell 1996; 87:1171-80.
2. Thurston G, Suri C, Smith K, et al., Leakage-resistant blood vessels in mice transgenically overexpressing angiopoietin-1. Science 1999; 286:2511-4.
3. Maisonpierre P C, Suri C, Jones P F, et al., Angiopoietin-2, a natural antagonist for Tie2 that disrupts in vivo angiogenesis. Science 1997; 277:55-60
4. Hackett S F, Ozaki H, Strauss R W, et al., Angiopoietin 2 expression in the retina: upregulation during physiologic and pathologic neovascularization. J Cell Physiol 2000; 184: 275-84.
5. Patel J I, Hykin P G, Gregor Z J, et al., Angiopoietin concentrations in diabetic retinopathy. Br J Ophthalmol 2005; 89: 480-3.
6. Hackett S F, Wiegand S J, Yancopoulos G, Campochiaro P. Angiopoietin-2 plays an important role in retinal angiogenesis. J Cell Physiol 2002; 192:182-7.
7. Oshima Y, Deering T, Oshima S, et al., Angiopoietin-2 enhances retinal vessel sensitivity to vascular endothelial growth factor. J Cell Physiol 2004; 199:412-7.
8. Oshima Y, Oshima S, Nambu H, et al., Different effects of angiopoietin-2 in different vascular beds: new vessels are most sensitive. FASEB J 2005; 19:963-5.
9. Rangasamy S, Srinivasan R, Maestas J, et al., A potential role for angiopoietin 2 in the regulation of the blood-retinal barrier in diabetic retinopathy. Invest Ophthalmol Vis Sci 2011; 52: 3784-91.
10. Kajiya, K. Kidoya, H., Sawane, M., Matsumoto-Okazaki, Y., Yamanishi, H., Furuse, M., and Takakura, N. Promotion of Lymphatic Integrity by Angiopoietin-1/Tie2 Signaling during Inflammation. Am J Pathol 2012, 180:1273-1282; DOI: 10.1016/j.ajpath.2011.11.008
11. Yuan, H T, Khankin, E V, Karunmanchi, S A, Parikh, S M. Angiopoietin 2 Is a Partial Agonist/Antagonist of Tie2 Signaling in the Endothelium Mol Cell Biol. 2009 April; 29(8):2011-22. doi: 10.1128/MCB.01472-08. Epub 2009 Feb. 17.

### Example 2: AXT107 Potentiates the Activation of Tie2 by Ang2

In this example, a regulatory function of integrins on Ang-Tie signaling was investigated using an exemplary integrin-binding, biomimetic peptide, AXT107.

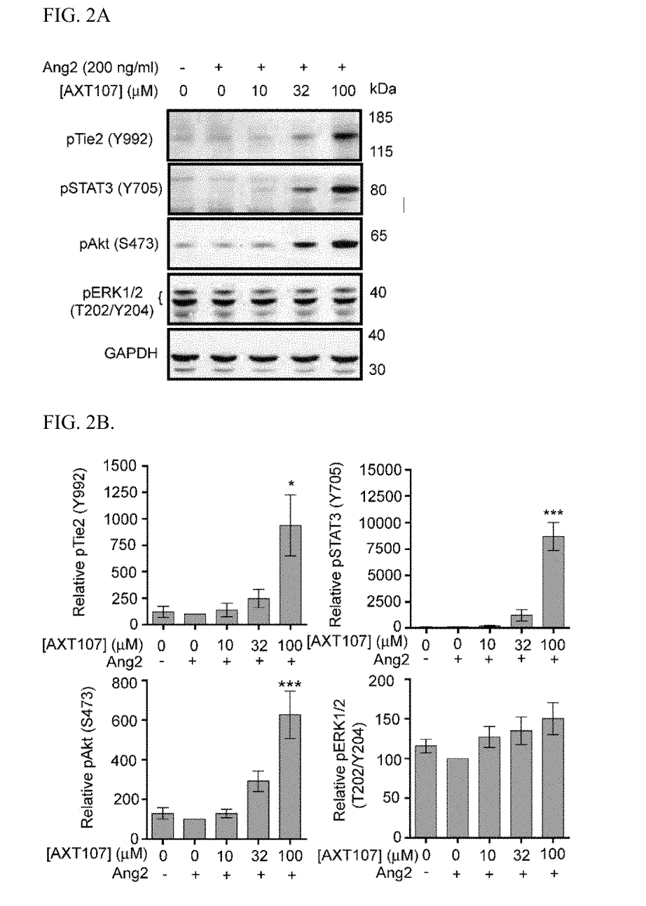

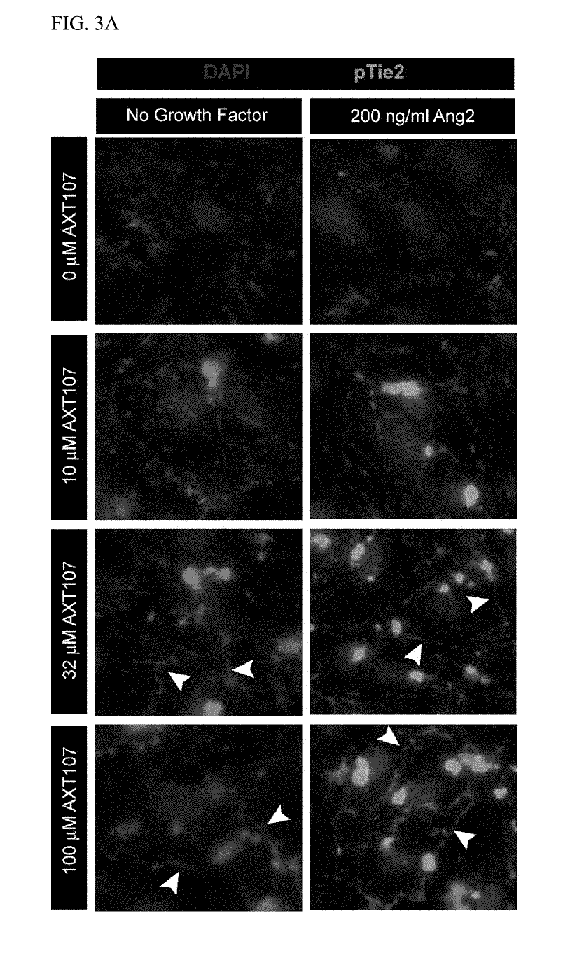

To investigate the effects of the integrin inhibitor AXT107 on Tie2 signaling, confluent monolayers of microvascular endothelial cells (MECs) on fibronectin-coated dishes were treated with various concentrations of AXT107 followed by exposure to the Tie2 ligands Ang1 or Ang2. Ang1 alone induced phosphorylation of Tie2 (data not shown) whereas Ang2 showed insignificant effects by itself (FIG. 2A and FIG. 2B).

Surprisingly, whereas AXT107 treatment did not significantly influence Ang1 activity (data not shown), a significant, dose-dependent increase in Tie2 phosphorylation was observed in cells treated in combination with AXT107 and Ang2 (FIG. 2A, first row, and FIG. 2B, top left graph). The phosphorylation of the downstream, pro-survival effectors STAT3 and Akt also increased with Ang2 and AXT107 treatment (FIG. 2A, second and third rows, and FIG. 2B, top right and bottom left graphs). However, the phosphorylation of proliferation-associated factor Erk1/2 remained constant in all tested conditions (FIG. 2A, fourth row and FIG. 2B, bottom right graph). For all cases, the total protein levels of Tie2 and the downstream targets remained unchanged (data not shown) and phosphorylation was not induced by peptide alone in absence of either ligand.

Integrin inhibition by AXT107 significantly decreases receptor phosphorylation and downstream signals for many RTKs, e.g., VEGFR2, c-Met, IGF1R, and PDGFRβ, as well as reduced total receptor levels through increased receptor degradation (Lee et al., Sci Rep. 2014; 4:7139). In contrast, AXT107 clearly potentiates the activation of Tie2 by Ang2 both in vitro and in vivo and does not influence total levels of Tie2, suggesting that increased degradation of the receptor does not occur for Tie2 as it does with other RTKs.

These data demonstrate that treatment with the exemplary integrin-binding, biomimetic peptide potentiates the normally weak agonistic activity of Ang2 towards Tie2, or converts Ang2 from Tie2 antagonist to agonist. Accordingly, AXT107 specifically activates downstream targets associated with endothelial cell (EC) survival and barrier function.

### Example 3: Changes in Tie2 Cellular Distribution Mediated by AXT107 Influences Receptor Activation

The discovery that AXT107 potentiates Ang2-mediated phosphorylation of Akt and STAT3 but does not potentiate ERK1/2 phosphorylation, suggests that AXT107 specifically activates junctional Tie2 rather than Tie2 molecules at the cell-extracellular matrix (ECM) interface. As such, subsequent experiments evaluate the effects of AXT107 at cell-cell junctions in MEC monolayers using immunofluorescence microscopy rather than at the EC-ECM interface.

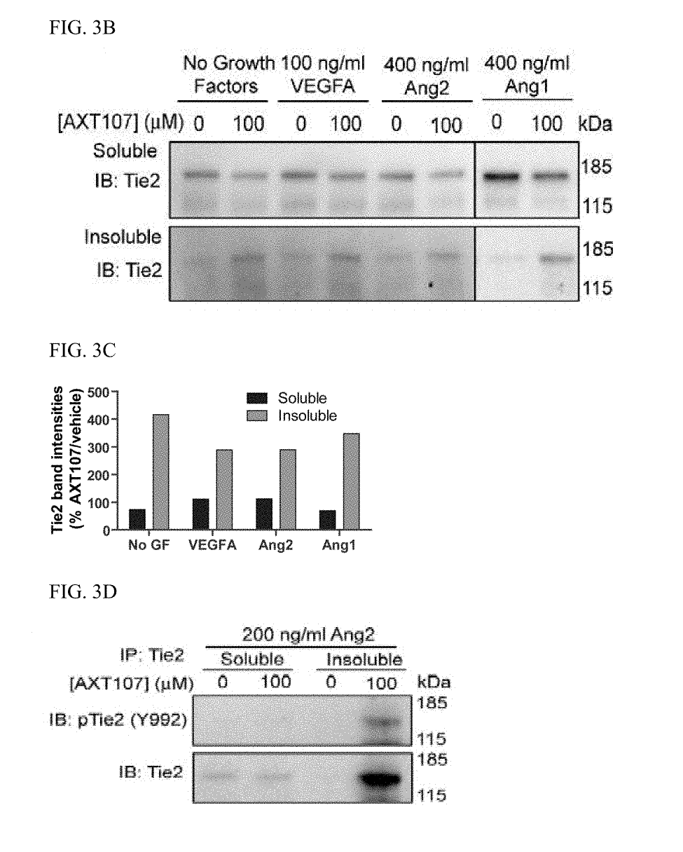

AXT107 was found to self-assemble into peptide complexes when added to media; this behavior is similar to the depots observed in mouse eyes (data not shown). In samples treated with Ang2 alone (FIG. 3A, top row), phospho-Tie2 was predominantly found in weak, punctate distributions across the cell surface. Samples treated with Ang2 and AXT107 had increased overall fluorescence intensity and redistributed phospho-Tie2 along cell-cell junctions and into large clusters that co-localized with the AXT107 peptide complexes (FIG. 3A, bottom three rows). These large clusters of phospho-Tie2 are unlikely the result of non-specific interactions between the AXT107 peptide complexes and the tested antibodies as no green fluorescence signal was observed for peptide complexes in regions devoid of cells or in wells treated with secondary antibodies alone (data not shown). Previous reports have emphasized the importance of clustering in Ang2's activation of Tie2 and may explain the potentiation of Tie2 phosphorylation by Ang2 and the activation of downstream effectors of vessel stability and quiescence.

Tie2 at EC-EC junctions form actin-rich complexes that are insoluble in Triton X-100-based lysis buffers but are soluble when distributed over the surface of the cell. Therefore, MEC monolayers were treated with various combinations of AXT107, Ang1, and Ang2 and cell lysates were fractionated by their solubility in Triton X-100-based lysis buffers. Experiments including VEGF 165 were also performed since VEGFR2 signaling often opposes the activities of Tie2. In each experiment, 100 μM AXT107 was used since it provided clear results relative to lower concentrations of AXT107.

Consistent with the observations from the immunofluorescence assays (as shown in FIG. 3A), increased amounts of Tie2 were found in the insoluble fraction of lysates treated with AXT107; the increased amounts were independent of the specific growth factor treatment (FIGS. 3B and 3C). Similar results were also obtained for Tie1 (data not shown), a co-receptor recently shown to be essential for the activation of junctional Tie2 (Korhonen et al., J Clin Invest. 2016; 126(9):3495-3510).

Next, experiments were performed to determine whether or not relocation of Tie2 to the insoluble fraction was important for Tie2's activation by Ang2. Tie2 was immunoprecipitated from fractionated MEC lysates exposed to Ang2 with or without AXT107 and then immunoblotted for phospho-Tie2. Interestingly, phosphorylation was observed only in the insoluble fractions of peptide-treated samples (FIG. 3D).

These data demonstrate that treatment with AXT107 peptide results in the translocation of Tie2 to EC-EC junctions and activation of Tie2.

### Example 4: AXT107 Disrupts Interactions Between α5 and β1 Integrin Subunits

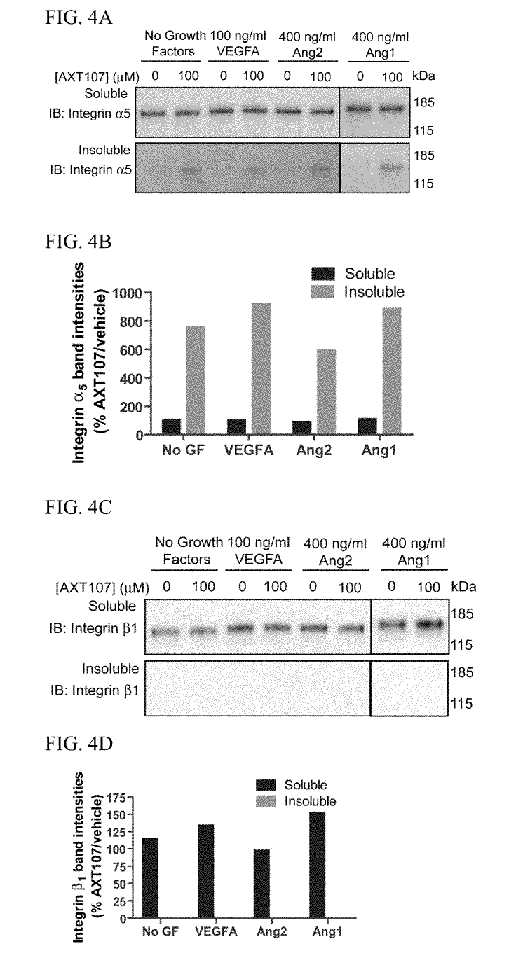

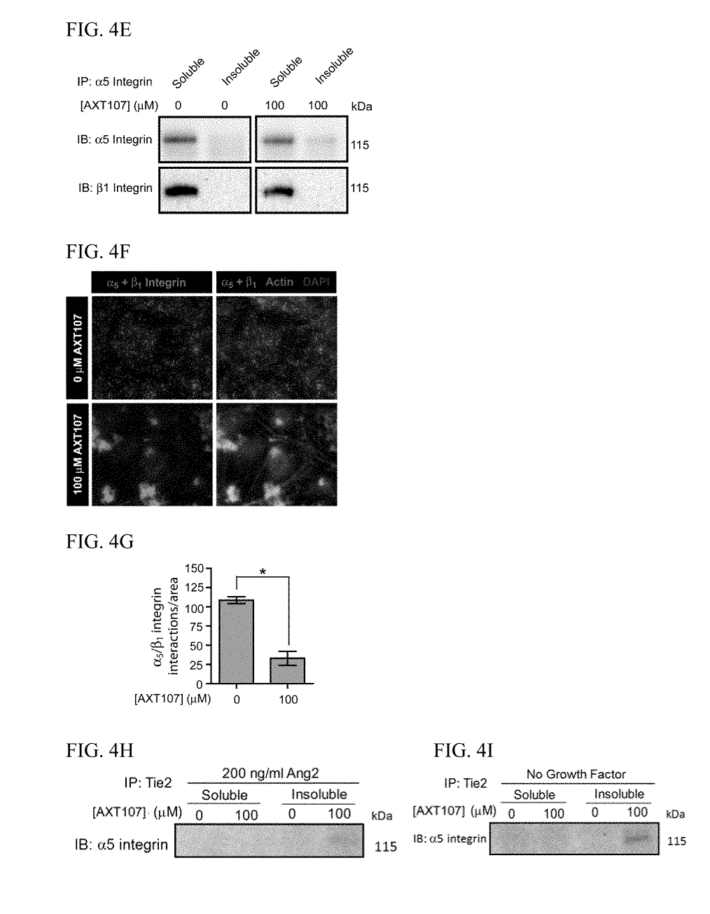

α₅β₁-integrin heterodimer and αᵥβ₃-integrin heterodimer are primary targets of AXT107. To investigate the possibility of an integrin-mediated mechanism for regulating Tie2, fractionated MEC lysates immunoblotted for the α₅ integrin subunit revealed that a portion of the α₅ integrin subunit relocated to the insoluble fraction in samples treated with AXT107 (FIGS. 4A and 4B); this result is similar to what was observed for both Tie2 (FIG. 3D) and Tie1 (data not shown).

Surprisingly, the β₁ integrin subunit was never observed in the insoluble fraction despite the use of long exposure times and high antibody concentrations (FIG. 4C to 4E). This suggests treatment with AXT107 disrupts the interaction between the as integrin subunit and β₁ integrin subunit in an integrin heterodimer. Since β₁ integrin is the only known β subunit to heterodimerize with the α₅ integrin subunit, it unlikely that the as subunit, that was observed here in the insoluble fractions, originated from a heterodimeric integrin pair other than α₅β₁. Unfortunately, high background impaired the visualization of α₅ integrin alone by immunofluorescence. Given the unexpected discovery that AXT107 dissociates the integrin heterodimer, this discovery was confirmed in several independent assays. Immunoprecipitation of α₅ integrin in Triton X-100 fractionated lysates revealed that while α₅ integrin could be observed in the insoluble fraction after AXT107 treatment, interactions with β₁ were only found in the soluble fractions (data not shown). Thus, interactions between Tie2 and heterodimerized α₅β₁ integrin appear to retain Tie2 at the EC-ECM interface. This is consistent with reports that Tie2 does not interact with (31 integrin in absence of the α5 subunit (Cascone et al., J Cell Biol. 2005; 170(6):993-1004); disruption of these integrin heterodimers allow for the formation of Tie2-containing complexes at EC-EC junctions and in large clusters.

Additionally, changes in the interaction between the α₅ s and β₁ subunits were further investigated using Duolink™ technology which can visualize individual interactions between α₅ and β₁ integrin subunits as distinct spots by fluorescence microscopy. Consistent with the results from the Triton X-100 fractionation studies (FIGS. 4A to 4E), interactions between α₅ and β₁ integrin subunits were significantly reduced in monolayers treated with AXT107 compared to vehicle alone (FIG. 4F and FIG. 4G).

Finally, it was determined whether or not the α₅ integrin subunit remains complexed with Tie2 following its disassociation with β₁ integrin. As shown in FIG. 4H and FIG. 4I, α₅ integrin was observed in the peptide-treated, insoluble fraction following immunoprecipitation of Tie2.

These data demonstrate that treatment with the AXT107 peptide dissociate α5 integrin and β1 integrin subunits from a heterodimer.

Interestingly, the knockdown of β1 integrin has been shown to decrease Akt phosphorylation; this contrasts the results from α5 integrin knockdown or peptide treatment. As a possible explanation, β1 is the most promiscuous of the integrins and decreases in its protein levels may impact more cellular activities, both Tie2-dependent and Tie2-independent, in comparison to knockdowns of the relatively-specific α5 integrin or conditions in which integrin levels remain constant but are inhibited. Taken together, these findings emphasize the importance of integrins in the preferential activation of signaling pathways downstream of Tie2.

### Example 5: Treatment with AXT107 Strengthens and Narrows Endothelial Cell Junctions

Having demonstrated that AXT107 potentiates the activation of Tie2 through the disruption of interactions between subunits in α₅β₁ integrin, the following experiments were performed to determine the functional consequence of this activity.

Tie2 signaling is a major regulator of vascular permeability and dysfunction in this activity is known to contribute to increased macular edema and disease progression. Specifically, Tie2 strengthens cell-cell junctions through the formation of trans interactions with Tie2 receptors on adjacent cells and the reorganization of VE-Cadherin complexes continuously along cell-cell junctions.

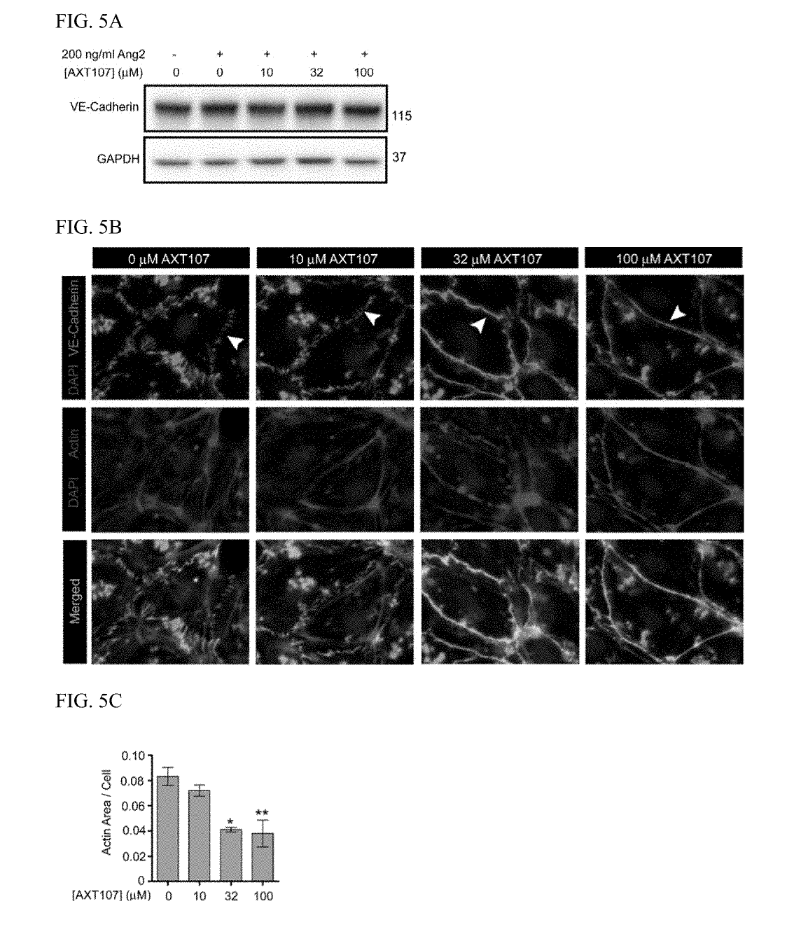

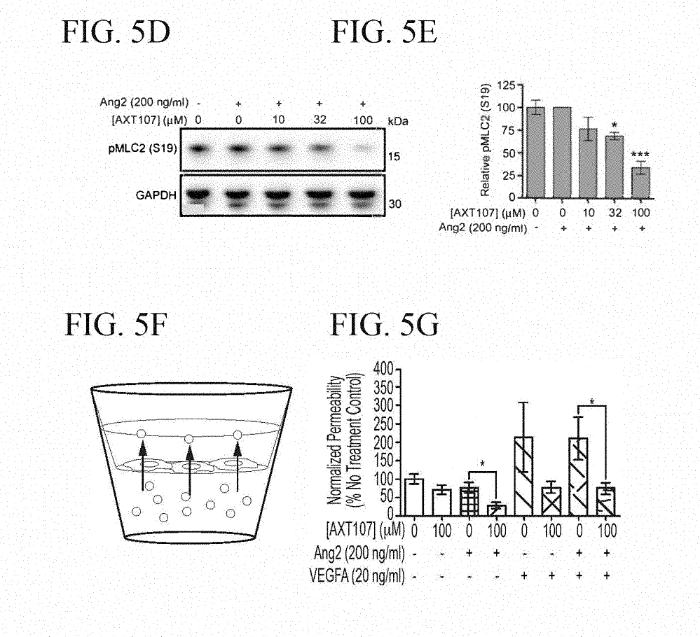

Consistent with previous reports of Tie2 activation by Ang1, the total level of VE-cadherin remained unchanged after three hours of AXT107 and Ang2 treatment (FIG. 5A). However, immunofluorescence imaging revealed clear changes in the structure of VE-cadherin junctions. As shown in FIG. 5B, at lower concentrations, the distribution of VE-cadherin was discontinuous and jagged in appearance but became progressively smoother with increasing concentrations of AXT107. The jaggedness of these junctions is related to the structure of actin within the cell. Absent AXT107 treatment (FIG. 5B, left column), radial actin fibers were arranged across the cells but became more cortical with increasing concentrations of AXT107 (compare with remaining columns in FIG. 5B; see, also, FIG. 5C). Radial actin functions to pull cells apart to increase permeability whereas junctional actin does not exert the same pull and thus results in decreased vascular permeability.

Phosphorylation of Tie2 is known to stimulate the Rap1-GTPase pathway, leading to a reduction in the phosphorylation of the downstream motor protein myosin light chain 2 (MLC2) associated with actin rearrangement. As shown in FIG. 5D, phosphorylation of MLC2 is reduced in a dose-dependent manner following treatment with AXT107 and Ang2.

The reorganization of VE-cadherin, actin, and Tie2 at endothelial cell junctions by AXT107 suggests that treatment with the peptide stabilizes cell-cell interactions. The integrity of these junctions is also important for the regulation of monolayer permeability by controlling the size of intercellular openings. The effect of the AXT107 on EC permeability was further investigated by the transendothelial diffusion of FITC-labeled dextran across MEC monolayers seeded onto permeable Transwell® substrates. A schematic of the assay is shown in FIG. 5F.

As shown in FIG. 5G, treatment with Ang2 or AXT107 alone influenced FITC-dextran diffusion across the monolayer whereas VEGF treatment appeared to increase permeability (although non-significantly) alone or in combination with Ang2. Interestingly, the addition of Ang2 alone or in combination with VEGF to monolayers pre-incubated with 100 μM AXT107 showed a significant decrease in FITC-dextran diffusion into the top chamber when compared to cells treated with the growth factors alone. A similar, but non-significant trend was also observed between monolayers treated in VEGF in the presence and absence of peptide.

These data demonstrate that treatment with the AXT107 peptide results in decreased monolayer permeability through the reorganization of F-actin and VE-cadherin.

The importance of integrin interactions in the regulation of Tie2 signaling suggests that natural mechanisms may exist for this to occur within organisms. The treatment of ECs with cartilage oligomeric matrix protein (COMP)-Ang1 has been shown to induce junctional relocation of Tie2, suggesting that it may stimulate Tie2's dissociation from β1 integrin through a yet-unknown mechanism. Interestingly, differences in the C-termini of Ang1 and Ang2 were found to alter their interactions with β1 integrin which, consequently, could only be activated by Ang2.

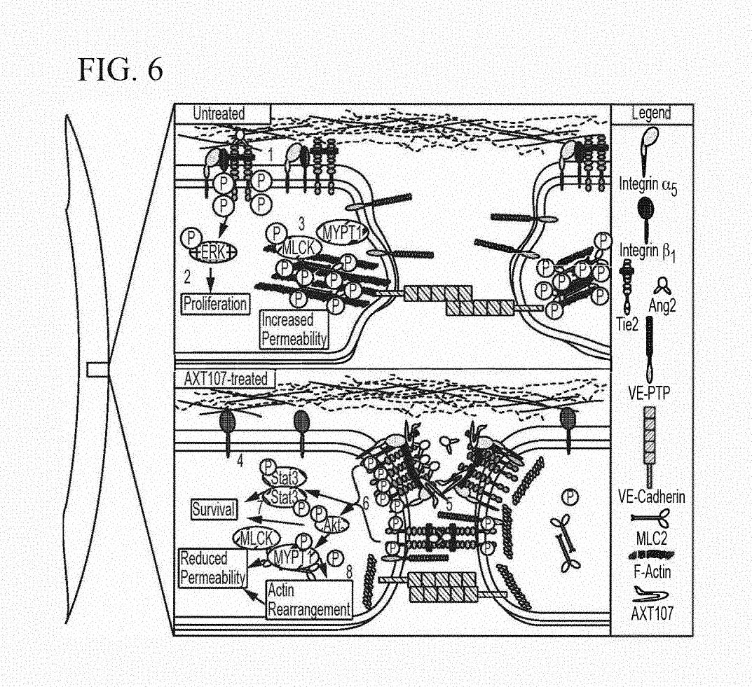

Without wishing to be bound by theory, the data disclosed herein provide a model for AXT107-mediated activation of Tie2. As illustrated in FIG. 6, top, in the absence of AXT107 (1) Ang2 weakly activates Tie2 in complex with integrin α₅β₁ heterodimers at the EC-ECM interface, which (2) preferentially activates proliferative signals (e.g., ERK1/2). (3) Active MLC kinase (MLCK) activates MLC3 and leads to formation of radial actin stress fibers within the cell and tension at EC-EC cell junctions. However, in the presence of AXT107, in FIG. 6, bottom, (4) α₅ integrin separates from β₁ integrin and (5) migrates to EC-EC junctions along with Tie2 to form large complexes and/or trans-interactions across junctions. (6) These complexes potentiate the phosphorylation of Tie2 and activate (7) Akt- and STAT3-mediated survival pathways. Additionally, (8) MLC phosphatase is activated via a RAP1 or RAC1 pathway, which leads to reduced MLC2 activity, increased cortical actin, and stabilized junctions.

### Example 6: In Vivo, the Exemplary Integrin-Binding, Biomimetic Potentiates Tie2 Phosphorylation

To investigate the effects of AXT107 on Tie2 activation in vivo, retinopathy of prematurity (ROP) mouse model was used. In this system, P7 pups are placed in 75% 02 for five days, resulting in a loss of retinal capillary density from hyperoxia and a rapid induction of neovascularization upon the pup's return to normoxic conditions. Here, the in vivo potential of the peptide was demonstrated using an eye vasculature model. Increased levels of Ang2 in the vitreous contribute to vessel leakage and macular edema in various retinopathies.

The Following Exemplary Methods and Materials were Used the Above Examples:

Cell Culture and Reagents

Human dermal microvascular endothelial cells (Lonza) were maintained at 37° C. and 5% CO₂ in EBM-2MV medium (Lonza) and used between passages two through seven. Where applicable, cells were serum starved in EBM-2 medium (Lonza) with no supplements. For FITC-Dextran permeability assays phenol red-free media were used to avoid auto-fluorescence. AXT107 were manufactured at New England Peptide by solid state synthesis, lyophilized, and dissolved in 100% DMSO. After dilution, preferably, DMSO concentrations did not exceed 0.25%.

Western Blotting

For Ang1/2 signaling investigations, cell culture dishes (10 cm diameter) were coated with 5 μg/ml fibronectin (FN1; Sigma-Aldrich, St. Louis, Mo.) for two hours at 37° C. The FN1 solution was then removed by aspiration and 5×10⁶ microvascular endothelial cells (MECs; Lonza, Walkersville, Md.) were plated in EGM-2MV media (Lonza) and cultured for forty-eight hours at 37° C. The cells were then serum starved for sixteen hours in serum-free EGM-2 base media (Lonza). AXT107 (0-100 μM, as indicated) was subsequently added to each culture and incubated for seventy-five minutes at 37° C. The cultures were then treated with 1 mM sodium vanadate (New England Biolabs, Ipswich, Mass.) for fifteen minutes to enhance the phospho-Tie2 signal followed by stimulation with 200 ng/ml angiopoietin (R&D Systems, Minneapolis, Minn.) for an additional fifteen minutes. The cells were then transferred to ice, washed twice with ice cold Dulbecco's phosphate-buffered saline (dPBS) containing Ca²⁺ and Mg²⁺, and collected by scraping in 500 μl of 1×Blue Loading Buffer (Cell Signaling, Danvers, Mass.). Lysate samples were then sonicated, boiled, and resolved by SDS-PAGE. Specific proteins were identified by Western blot, using the following primary antibodies: Cell Signaling—phospho-Tie2 (Y992) (Cat#: 4221), Tie2 (Cat#: 7403), phospho-Stat3 (Y705) (Cat#: 4113), Stat3 (Cat#: 4904), phospho-Akt (S473) (Cat#: 4058), Akt (Cat#: 9272), phospho-p44/42 MAPK (T202/Y204) (Cat#: 4370), p44/42 MAPK (Cat#: 4695); BD Transduction Laboratories—β₁ integrin (Cat#: 610467); Millipore—α₅ integrin (Cat#: AB1928) and detected with HRP-conjugated goat anti-rabbit and sheep anti-mouse secondary antibodies (GE healthcare).

Triton X-100 Fractionation

The isolation of Triton X-100 soluble and insoluble fractions was performed using modifications to previously-described procedures (see, e.g., Lampugnani et al., J Cell Biol. 1995; 129(1):203-217). FN1-coated six-well plates were seeded with 2.5×10⁶ cells and cultured for forty-eight hours, as described above. The cultures were then serum starved for ninety minutes in EBM-2 media, treated with 100 μM AXT107 or DMSO vehicle, and fifteen minutes with 1 mM sodium vanadate. The cells were then stimulated with either 100 ng/ml VEGFA, 400 ng/ml Ang2, or PBS for fifteen minutes. The plates were then transferred to ice and washed twice with cold dPBS containing Ca²⁺ and Mg²⁺ and twice with EBM-2 media. The media was then removed and the cells incubated for thirty minutes on ice, at 4° C. in 200 μl of Triton X-100 extraction buffer (10 mM Tris-HCl, pH 7.5; 150 mM NaCl; 2 mM CaCl₂); 1% NP-40; 1% Triton C-100; and a protease inhibitor cocktail (Cell Signaling, Cat#: 5871)) with occasional agitation. The extraction buffer was gently collected and centrifuged at 12,000×g for five minutes. The supernatant was then mixed with 125 μl of 3×Blue Loading Dye, boiled, and saved as the Triton X-100 soluble fraction at −20° C. The remaining insoluble fraction was washed twice with wash buffer (10 mM Tris-HCl, pH 7.5; 150 mM NaCl; cOmplete™ Mini protease inhibitor tablets (Roche)) and collected in 375 μl of 1×Blue Loading Dye with scraping followed by centrifugation and boiling, as described above. This lysate was saved at −20° C. as the Triton X-100 insoluble fraction. Samples were analyzed by western blot as described above.

For pull-down variations, insoluble fractions were instead collected in RIPA buffer (Sigma) treated with a protease and phosphatase inhibitor cocktail (Cell Signaling) and 5 mM EDTA. Lysates were sonicated briefly and incubated for one hour with anti-Tie2 (Cell Signaling, Cat#: 4224) or anti-α₅ integrin (Millipore; Cat#: AB1928) with end-over-end mixing. Subsequently, 20 μl of Protein Agarose A/G beads (Santa Cruz) were added and the samples incubated for another hour. Beads were collected by centrifugation at 1,500×g and 4° C., washed four times with PBS, resuspended in SDS-based Blue Loading Dye (Cell Signaling), boiled and resolved by SDS-PAGE.

Immunofluorescence

Glass-bottomed, 96-well plates with half well size were coated with 10 μg/ml FN1 for two hours at 37° C. The FN1 solution was then removed by aspiration and the plate seeded with 4×10³ MECs in EGM-2MV media. After twenty-four hours, the cells were washed once with dPBS containing Ca²⁺ and Mg²⁺ to remove dead cells and the cells were allowed to grow for an additional twenty-four hours. For three-hour duration treatments (i.e., VE-cadherin), cells were washed twice with dPBS containing Ca²⁺ and Mg²⁺ and serum starved in EBM-2 for ninety minutes. The media was then removed and the cells were treated for three hours with 100 μl of EBM-2 media containing 200 ng/ml Ang2 or PBS and varying concentrations of AXT107 or DMSO. For fifteen minute-treated samples (i.e., phospho-Tie2), the cells were serum starved in EBM-2 media for 165 minutes, incubated for ninety minutes with varying concentrations of AXT107 or DMSO in EBM-2, and finally stimulated for fifteen minutes with 200 ng/ml Ang2 or PBS supplemented with peptide to retain the same concentrations. These times were chosen so that both treatment procedures would be completed at the same time. The cells were then washed twice with cold dPBS containing Ca²⁺ and Mg²⁺ and fixed in 10% neutral buffered formalin for fifteen minutes. The formalin solution was then removed, the wells washed three times in dPBS containing Ca²⁺ and Mg²⁺. The cells were then blocked in blocking buffer (5% normal goat serum; 0.3% Triton X-100 in dPBS containing Ca²⁺ and Mg²⁺) and stained for sixteen hours with primary antibodies for phospho-Tie2 (Y992) (R&D Systems; Cat#: AF2720) or VE-cadherin (Cell Signaling; Cat#: 2500) diluted 1:150 in antibody dilution buffer (1% BSA; 0.3% Triton X-100 in dPBS containing Ca²⁺ and Mg²⁺). The wells were then washed three times with dPBS and incubated for one hour with Alexafluor 488-conjugated goat anti-rabbit secondary antibodies (Cell Signaling; Cat#: 4412) diluted 1:300 in antibody dilution buffer. The wells were then washed twice and stained for twenty minutes with Alexafluor 555-conjugated phalloidin (Cell Signaling; Cat#: 8953) diluted 1:20 in PBS. The cells were then washed twice again in dPBS, stained with DAPI for twenty minutes, and solution exchanged with dPBS for imaging. Cells were imaged using the BD Pathway 855 system and Attovision software (BD Biosciences).

Duolink Protein Interaction Analysis

Glass bottom, 96-well plates were coated with FN1, seeded with MECs, as described above for the immunofluorescence experiments. After growing for forty-eight hours, cells were serum starved for three hours, treated with 100 μM AXT107 or DMSO vehicle for ninety minutes, washed twice with dPBS containing Ca²⁺ and Mg²⁺, and fixed in 10% neutral buffered formalin. Cells were blocked in 5% normal goat serum; 0.3% Triton X-100 in dPBS containing Ca²⁺ and Mg²⁺ for one hour and incubated overnight at 4° C. with rabbit anti-α5 integrin and mouse anti-β1 integrin antibodies in PBS containing Ca²⁺ and Mg²⁺ with 1% BSA and 0.1% Triton X-100. Interaction spots were developed using DUOLINK green detection reagent according to the manufacturer's instructions and detected using the BD pathway 855 system.

FITC Transwell Permeability Assay

Transwell, twenty-four-well inserts (Corning) were coated with 7.5 μg/cm² FN1 for two hours at 37° C., aspirated, and then dried for thirty minutes at room temperature. Wells were then seeded with 7.5×10⁴ MECs in 100 μl of EBM-2 media (without phenol red) and allowed to settle for thirty minutes at room temperature. 1 ml of EGM-2 media was then added to the bottom chamber and an additional 200 μl to the top chamber. The plate was incubated for twenty-four hours at 37° C. after which the media was aspirated and an additional 7.5×10⁴ MECs were plated in each well as described above. After forty-eight hours at 37° C., the media was aspirated from both chambers and the cells were washed twice in dPBS containing Ca²⁺ and Mg²⁺, once with EBM-2 media (without phenol red) and serum starved in EBM-2 media applied to both chambers for two hours at 37° C. After this incubation, 100 μM AXT107 or an equivalent amount of DMSO vehicle was added and incubated for an additional ninety minutes. In the top chamber, the cells were then treated with 200 ng/ml Ang2, 100 ng/ml VEGFA, both, or PBS control and in the bottom chamber, the cells were then treated with 25 μg/ml FITC-Dextran (40 kDa MW). AXT107 was also added in both chambers to maintain a concentration of 100 μM. After three hours, 10 μl was removed from the top chamber of each well and mixed with 90 μl of water in a clear bottom, 96-well plate. Fluorescence values for each sample were calculated using a Perkin Elmer plate reader.

### REFERENCES

Eklund L, Kangas J and Saharinen P. Angiopoietin-Tie signalling in the cardiovascular and lymphatic systems. Clin Sci (Lond). 2017; 131(1):87-103.
Saharinen P, Eklund L and Alitalo K. Therapeutic targeting of the angiopoietin-TIE pathway. Nat Rev Drug Discov. 2017.
Davis S, Aldrich T H, Jones P F, Acheson A, Compton D L, Jain V, Ryan T E, Bruno J, Radziejewski C, Maisonpierre P C and Yancopoulos G D. Isolation of angiopoietin-1, a ligand for the TIE2 receptor, by secretion-trap expression cloning. Cell. 1996; 87(7):1161-1169.
Saharinen P, Eklund L, Miettinen J, Wirkkala R, Anisimov A, Winderlich M, Nottebaum A, Vestweber D, Deutsch U, Koh G Y, Olsen B R and Alitalo K. Angiopoietins assemble distinct Tie2 signalling complexes in endothelial cell-cell and cell-matrix contacts. Nat Cell Biol. 2008; 10(5):527-537.
Frye M, Dierkes M, Kuppers V, Vockel M, Tomm J, Zeuschner D, Rossaint J, Zarbock A, Koh G Y, Peters K, Nottebaum A F and Vestweber D. Interfering with VE-PTP stabilizes endothelial junctions in vivo via Tie-2 in the absence of VE-cadherin. J Exp Med. 2015; 212(13):2267-2287.
Dalton A C, Shlamkovitch T, Papo N and Barton W A. Constitutive Association of Tie1 and Tie2 with Endothelial Integrins is Functionally Modulated by Angiopoietin-1 and Fibronectin. PLoS One. 2016; 11(10):e0163732.
Fiedler U, Scharpfenecker M, Koidl S, Hegen A, Grunow V, Schmidt J M, Kriz W, Thurston G and Augustin H G. The Tie-2 ligand angiopoietin-2 is stored in and rapidly released upon stimulation from endothelial cell Weibel-Palade bodies. Blood. 2004; 103(11):4150-4156.
Maisonpierre P C, Suri C, Jones P F, Bartunkova S, Wiegand S J, Radziejewski C, Compton D, McClain J, Aldrich T H, Papadopoulos N, Daly T J, Davis S, Sato T N and Yancopoulos G D. Angiopoietin-2, a natural antagonist for Tie2 that disrupts in vivo angiogenesis. Science. 1997; 277(5322):55-60.
Benest A V, Kruse K, Savant S, Thomas M, Laib A M, Loos E K, Fiedler U and Augustin H G. Angiopoietin-2 is critical for cytokine-induced vascular leakage. PLoS One. 2013; 8(8):e70459.
Tabruyn S P, Colton K, Morisada T, Fuxe J, Wiegand S J, Thurston G, Coyle A J, Connor J and McDonald D M. Angiopoietin-2-driven vascular remodeling in airway inflammation. Am J Pathol. 2010; 177(6):3233-3243.
Daly C, Pasnikowski E, Burova E, Wong V, Aldrich T H, Griffiths J, loffe E, Daly T J, Fandl J P, Papadopoulos N, McDonald D M, Thurston G, Yancopoulos G D and Rudge J S. Angiopoietin-2 functions as an autocrine protective factor in stressed endothelial cells. Proc Natl Acad Sci USA. 2006; 103(42):15491-15496.
Yuan H T, Khankin E V, Karumanchi S A and Parikh S M. Angiopoietin 2 is a partial agonist/antagonist of Tie2 signaling in the endothelium. Mol Cell Biol. 2009; 29(8):2011-2022.
Korhonen E A, Lampinen A, Giri H, Anisimov A, Kim M, Allen B, Fang S, D'Amico G, Sipila T J, Lohela M, Strandin T, Vaheri A, Yla-Herttuala S, Koh G Y, McDonald D M, Alitalo K, et al., Tie1 controls angiopoietin function in vascular remodeling and inflammation. J Clin Invest. 2016; 126(9):3495-3510.
Shen J, Frye M, Lee B L, Reinardy J L, McClung J M, Ding K, Kojima M, Xia H, Seidel C, Lima e Silva R, Dong A, Hackett S F, Wang J, Howard B W, Vestweber D, Kontos C D, et al., Targeting V E-PTP activates TIE2 and stabilizes the ocular vasculature. J Clin Invest. 2014; 124(10):4564-4576.
Singh H, Milner C S, Aguilar Hernandez M M, Patel N and Brindle N P. Vascular endothelial growth factor activates the Tie family of receptor tyrosine kinases. Cell Signal. 2009; 21(8):1346-1350.
Cascone I, Napione L, Maniero F, Serini G and Bussolino F. Stable interaction between alpha5beta1 integrin and Tie2 tyrosine kinase receptor regulates endothelial cell response to Ang-1. J Cell Biol. 2005; 170(6):993-1004.
Lee E, Lee S J, Koskimaki J E, Han Z, Pandey N B and Popel A S. Inhibition of breast cancer growth and metastasis by a biomimetic peptide. Sci Rep. 2014; 4:7139.
Karagiannis E D and Popel A S. A systematic methodology for proteome-wide identification of peptides inhibiting the proliferation and migration of endothelial cells. Proc Natl Acad Sci USA. 2008; 105(37):13775-13780.
Chen T T, Luque A, Lee S, Anderson S M, Segura T and Iruela-Arispe M L. Anchorage of VEGF to the extracellular matrix conveys differential signaling responses to endothelial cells. J Cell Biol. 2010; 188(4):595-609.
Soldi R, Mitola S, Strasly M, Defilippi P, Tarone G and Bussolino F. Role of alphavbeta3 integrin in the activation of vascular endothelial growth factor receptor-2. EMBO J. 1999; 18(4):882-892.
Veevers-Lowe J, Ball S G, Shuttleworth A and Kielty C M. Mesenchymal stem cell migration is regulated by fibronectin through alpha5beta1-integrin-mediated activation of PDGFR-beta and potentiation of growth factor signals. J Cell Sci. 2011; 124(Pt 8):1288-1300.
Rahman S, Patel Y, Murray J, Patel K V, Sumathipala R, Sobel M and Wijelath E S. Novel hepatocyte growth factor (HGF) binding domains on fibronectin and vitronectin coordinate a distinct and amplified Met-integrin induced signalling pathway in endothelial cells. BMC Cell Biol. 2005; 6(1):8.
Baron V and Schwartz M. Cell adhesion regulates ubiquitin-mediated degradation of the platelet-derived growth factor receptor beta. J Biol Chem. 2000; 275(50):39318-39323.
Campochiaro P A, Khanani A, Singer M, Patel S, Boyer D, Dugel P, Kherani S, Withers B, Gambino L, Peters K, Brigell M and Group T-S. Enhanced Benefit in Diabetic Macular Edema from AKB-9778 Tie2 Activation Combined with Vascular Endothelial Growth Factor Suppression. Ophthalmology. 2016; 123(8):1722-1730.
Orfanos S E, Kotanidou A, Glynos C, Athanasiou C, Tsigkos S, Dimopoulou I, Sotiropoulou C, Zakynthinos S, Armaganidis A, Papapetropoulos A and Roussos C. Angiopoietin-2 is increased in severe sepsis: correlation with inflammatory mediators. Crit Care Med. 2007; 35(1):199-206.
Ziegler T, Horstkotte J, Schwab C, Pfetsch V, Weinmann K, Dietzel S, Rohwedder I, Hinkel R, Gross L, Lee S, Hu J, Soehnlein O, Franz W M, Sperandio M, Pohl U, Thomas M, et al., Angiopoietin 2 mediates microvascular and hemodynamic alterations in sepsis. J Clin Invest. 2013.
Han S, Lee S J, Kim K E, Lee H S, Oh N, Park I, Ko E, Oh S J, Lee Y S, Kim D, Lee S, Lee D H, Lee K H, Chae S Y, Lee J H, Kim S J, et al., Amelioration of sepsis by TIE2 activation-induced vascular protection. Sci Transl Med. 2016; 8(335):335ra355.
Lampugnani M G, Corada M, Caveda L, Breviario F, Ayalon O, Geiger B and Dejana E. The molecular organization of endothelial cell to cell junctions: differential association of plakoglobin, beta-catenin, and alpha-catenin with vascular endothelial cadherin (V E-cadherin). J Cell Biol. 1995; 129(1):203-217.

What is claimed is:

1. A method for preventing or treating a condition involving Tie-2-related vascular or lymphatic permeability in a patient, comprising: administering collagen IV-derived biomimetic peptide to said patient in an amount effective to reduce Tie-2-dependent vascular or lymphatic permeability.

2. The method of claim 1, wherein the condition is diabetic macular edema, retinal vein occlusion, wet age-related macular degeneration (wet AMD), background diabetic retinopathy, cancer, influenza, hemorrhagic fever, or cerebral malaria.

3. The method of claim 1, wherein the condition is tumor growth or metastasis.

4. The method of claim 1, wherein the condition is an inflammatory condition involving lymphatic dysfunction.

5. The method of claim 1, wherein the condition is vascular permeability prior to chemotherapy for cancer.

6. The method of claim 5, wherein the peptide is administered in an amount effective to normalize tumor vasculature, followed by administration of chemotherapy.

7. The method of claim 1, wherein the condition is lung cancer, which is optionally NSCLC or SCLC, liver cancer, triple-negative breast cancer, or glioblastoma.

8. The method of claim 1, wherein the condition is sepsis.

9. The method of claim 1, wherein the condition is capillary leak syndrome.

10. The method of claim 1, wherein the condition is an inflammatory condition of the lung, which is optionally acute respiratory distress syndrome, chronic asthma, or chronic obstructive pulmonary disorder (COPD).

11. The method of claim 1, wherein the condition is angioedema.

12. The method of claim 1, wherein the condition is vascular leak syndrome.

13. The method of claim 2, wherein a composition comprising the peptide of SEQ ID NOs: 1-4 is administered to a patient having diabetic macular edema, retinal vein occlusion, wet age-related macular degeneration (wet AMD), or background diabetic retinopathy, by intravitreal injection at a dose of from about 100 μg to about 1000 μg of the peptide, and with a frequency of injection of no more than monthly.

14. The method of claim 13, wherein the frequency of injection is no more than about every other month.

15. The method of claim 13, wherein the frequency of injection is no more than about every three months.

16. The method of claim 13, wherein the peptide is administered after unsuccessful VEGF blockade or inhibitor therapy.

17. The method of any one of claims 1 to 16, wherein the condition is refractory or only partially-responsive to VEGF blockade or inhibitor therapy.

18. The method of claim 17, wherein the peptide is administered after unsuccessful VEGF blockade or inhibitor therapy.

19. The method of claim 18, wherein the peptide is administered as an alternative to VEGF blockade or inhibitor therapy.

20. The method of claim 19, wherein the peptide is administered in combination with VEGF blockade therapy.

21. The method of any one of claims 1 to 20, wherein the peptide comprises the amino acid sequence of any one of SEQ ID NOs: 1-4.

22. The method of any one of claims 1 to 21, wherein the peptide is derived from the α5 fibril of collagen IV, or a biomimetic thereof.

23. The method of claim 22, wherein the peptide is:
(SEQ ID NO: 5)
LRRFSTMPFMF(Abu)NINNV(Abu)NF,
(SEQ ID NO: 6)
LRRFSTMPAMF(Abu)NINNV(Abu)NF,
(SEQ ID NO: 7)
LRRFSTMPFAF(Abu)NINNV(Abu)NF,
(SEQ ID NO: 8)
LRRFSTMPFMA(Abu)NINNV(Abu)NF,
(SEQ ID NO: 9)
LRRFSTMPF(Nle)F(Abu)NINNV(Abu)NF,
(SEQ ID NO: 10)
LRRFSTMPFM(4-ClPhe)(Abu)NINNV(Abu)NF,
(SEQ ID NO: 11)
LRRFSTMPFMFSNINNVSNF,
(SEQ ID NO: 12)
LRRFSTMPFMFANINNVANF,
(SEQ ID NO: 13)
LRRFSTMPFMFININNVINF,
(SEQ ID NO: 14)
LRRFSTMPFMFTNINNVTNF,
(SEQ ID NO: 15)
LRRFSTMPFMF(AllyGly)NINNV(AllyGly)NF ,
(SEQ ID NO: 16)
LRRFSTMPFMFVNINNVVNF,
(SEQ ID NO: 17)
LRRFSTMPFdAFININNVINF,
(SEQ ID NO: 18)
LRRFSTMPFAFININNVINF,
(SEQ ID NO: 19)
LRRFSTAPFAFININNVINF,
(SEQ ID NO: 20)
LRRFSTAPFdAFIDINDVINF,
(SEQ ID NO: 21)
LRRFSTAPFAFIDINDVINW,
(SEQ ID NO: 22)
dLRRdLRRFSTAPFAFIDINDVINF,
(SEQ ID NO: 23)
LRRFSTAPFAFIDINDVINdF,
or
(SEQ ID NO: 24)
dLRRFSTAPFAFIDINDVINdF.

24. The method of claim 22, wherein the peptide is:
(SEQ ID NO: 25)
F(Abu)NINNV(Abu)N,
(SEQ ID NO: 26)
FTNINNVTN,
(SEQ ID NO: 27)
FININNVINF,
(SEQ ID NO: 28)
FSNINNVSNF,
(SEQ ID NO: 29)
FANINNVANF,
(SEQ ID NO: 30)
F(AllyGly)NINNV(AllyGly)NF,
(SEQ ID NO: 31)
FVNINNVVNF,
(SEQ ID NO: 32)
FIDINDVINF,
(SEQ ID NO: 33)
FIDINDVINW,
(SEQ ID NO: 34)
FTDINDVTN,
(SEQ ID NO: 35)
A(Abu)NINNV(Abu)NF,
or
(SEQ ID NO: 36)
(4-ClPhe)(Abu)NINNV(Abu)NF.

25. The method of any one of claims 1 to 24, wherein the peptide is conjugated to, or loaded into, nanoparticles or microparticles.

26. The method of claim 25, wherein the nanoparticles or microparticles comprise PLGA-PEG.

27. A peptide or particle formulation thereof, the peptide having the amino acid sequence of any one of SEQ ID NOs: 1-36, and which is optionally a peptide having a sequence selected from SEQ ID NOs: 5 to 36.

28. The peptide or particle formulation of claim 27, wherein the formulation comprises from 100 μg to about 1000 μg of peptide agent.

29. The formulation of claim 28, wherein the formulation does not involve encapsulation into particles.

30. The peptide or particle formulation of claim 27, wherein the formulation comprises from about 1 mg to about 10 mg per dose.

31. The peptide or particle formulation of claim 30, wherein the formulation involves encapsulation into microparticles, optionally with free peptide.


SEQUENCE LISTING (36 sequences)

SEQ ID NO: 1 - protein, 20 aa, Artificial Sequence
  Synthetic Polypeptide
LRRFSTAPFAFIDINDVINF

SEQ ID NO: 2 - protein, 20 aa, Artificial Sequence
  Synthetic Polypeptide
  MISC_FEATURE (7)..(7): M, A, or G
  MISC_FEATURE (9)..(9): F, A, Y, or G
  MISC_FEATURE (10)..(10): M, A, G, D-Alanine, or norleucine
  MISC_FEATURE (11)..(11): F, A, Y, G, or 4-chlorophenylalanine
  MISC_FEATURE (12)..(12): Aminobutyric acid, G, S, A, V, T, I, L, or Allylglycine
  MISC_FEATURE (18)..(18): Aminobutyric acid, G, S, A, V, T, I, L, or Allylglycine
LRRFSTXPXXXXNINNVXNF

SEQ ID NO: 3 - protein, 20 aa, Artificial Sequence
  Synthetic Polypeptide
LRRFSTAPFAFIDINDVINF

SEQ ID NO: 4 - protein, 20 aa, Artificial Sequence
  Synthetic Polypeptide
  MISC_FEATURE (7)..(7): M, A, or G
  MISC_FEATURE (9)..(9): F, A, Y, or G
  MISC_FEATURE (10)..(10): M, A, G, D-Alanine, or norleucine
  MISC_FEATURE (11)..(11): F, A, Y, G, or 4-chlorophenylalanine
  MISC_FEATURE (12)..(12): Aminobutyric acid, G, S, A, V, T, I, L, or Allylglycine
  MISC_FEATURE (18)..(18): Aminobutyric acid, G, S, A, V, T, I, L, or Allylglycine
LRRFSTXPXXXXDINDVXNF

SEQ ID NO: 5 - protein, 20 aa, Artificial Sequence
  Synthetic Polypeptide
  MISC_FEATURE (12)..(12): 2-Aminobutyric acid
  MISC_FEATURE (18)..(18): 2-Aminobutyric acid
LRRFSTMPFMFXNINNVXNF

SEQ ID NO: 6 - protein, 20 aa, Artificial Sequence
  Synthetic Polypeptide
  MISC_FEATURE (12)..(12): 2-Aminobutyric acid
  MISC_FEATURE (18)..(18): 2-Aminobutyric acid
LRRFSTMPAMFXNINNVXNF

SEQ ID NO: 7 - protein, 20 aa, Artificial Sequence
  Synthetic Polypeptide
  MISC_FEATURE (12)..(12): 2-Aminobutyric acid
  MISC_FEATURE (18)..(18): 2-Aminobutyric acid
LRRFSTMPFAFXNINNVXNF

SEQ ID NO: 8 - protein, 20 aa, Artificial Sequence
  Synthetic Polypeptide
  MISC_FEATURE (12)..(12): 2-Aminobutyric acid
  MISC_FEATURE (18)..(18): 2-Aminobutyric acid
LRRFSTMPFMAXNINNVXNF

SEQ ID NO: 9 - protein, 20 aa, Artificial Sequence
  Synthetic Polypeptide
  MISC_FEATURE (10)..(10): norleucine
  MISC_FEATURE (12)..(12): 2-Aminobutyric acid
  MISC_FEATURE (18)..(18): 2-Aminobutyric acid
LRRFSTMPFXFXNINNVXNF

SEQ ID NO: 10 - protein, 20 aa, Artificial Sequence
  Synthetic Polypeptide
  MISC_FEATURE (11)..(11): 4-chlorophenylalanine
  MISC_FEATURE (12)..(12): 2-Aminobutyric acid
  MISC_FEATURE (18)..(18): 2-Aminobutyric acid
LRRFSTMPFMXXNINNVXNF

SEQ ID NO: 11 - protein, 20 aa, Artificial Sequence
  Synthetic Polypeptide
LRRFSTMPFMFSNINNVSNF

SEQ ID NO: 12 - protein, 20 aa, Artificial Sequence
  Synthetic Polypeptide
LRRFSTMPFMFANINNVANF

SEQ ID NO: 13 - protein, 20 aa, Artificial Sequence
  Synthetic Polypeptide
LRRFSTMPFMFININNVINF

SEQ ID NO: 14 - protein, 20 aa, Artificial Sequence
  Synthetic Polypeptide
LRRFSTMPFMFTNINNVTNF

SEQ ID NO: 15 - protein, 20 aa, Artificial Sequence
  Synthetic Polypeptide
  MISC_FEATURE (12)..(12): Allylglycine
  MISC_FEATURE (18)..(18): Allylglycine
LRRFSTMPFMFXNINNVXNF

SEQ ID NO: 16 - protein, 20 aa, Artificial Sequence
  Synthetic Polypeptide
LRRFSTMPFMFVNINNVVNF

SEQ ID NO: 17 - protein, 20 aa, Artificial Sequence
  Synthetic Polypeptide
  MISC_FEATURE (10)..(10): D-Alanine
LRRFSTMPFXFININNVINF

SEQ ID NO: 18 - protein, 20 aa, Artificial Sequence
  Synthetic Polypeptide
LRRFSTMPFAFININNVINF

SEQ ID NO: 19 - protein, 20 aa, Artificial Sequence
  Synthetic Polypeptide
LRRFSTAPFAFININNVINF

SEQ ID NO: 20 - protein, 20 aa, Artificial Sequence
  Synthetic Polypeptide
  MISC_FEATURE (10)..(10): D-Alanine
LRRFSTAPFXFIDINDVINF

SEQ ID NO: 21 - protein, 20 aa, Artificial Sequence
  Synthetic Polypeptide
LRRFSTAPFAFIDINDVINW

SEQ ID NO: 22 - protein, 23 aa, Artificial Sequence
  Synthetic Polypeptide
  MISC_FEATURE (1)..(1): D-Leucine
  MISC_FEATURE (4)..(4): D-Leucine
XRRXRRFSTAPFAFIDINDVINF

SEQ ID NO: 23 - protein, 20 aa, Artificial Sequence
  Synthetic Polypeptide
  MISC_FEATURE (20)..(20): D-Phenylalanine
LRRFSTAPFAFIDINDVINX

SEQ ID NO: 24 - protein, 20 aa, Artificial Sequence
  Synthetic Polypeptide
  MISC_FEATURE (1)..(1): D-Leucine
  MISC_FEATURE (20)..(20): D-Phenylalanine
XRRFSTAPFAFIDINDVINX

SEQ ID NO: 25 - protein, 9 aa, Artificial Sequence
  Synthetic Polypeptide
  MISC_FEATURE (2)..(2): 2-Aminobutyric acid
  MISC_FEATURE (8)..(8): 2-Aminobutyric acid
FXNINNVXN

SEQ ID NO: 26 - protein, 9 aa, Artificial Sequence
  Synthetic Polypeptide
FTNINNVTN

SEQ ID NO: 27 - protein, 10 aa, Artificial Sequence
  Synthetic Polypeptide
FININNVINF

SEQ ID NO: 28 - protein, 10 aa, Artificial Sequence
  Synthetic Polypeptide
FSNINNVSNF

SEQ ID NO: 29 - protein, 10 aa, Artificial Sequence
  Synthetic Polypeptide
FANINNVANF

SEQ ID NO: 30 - protein, 10 aa, Artificial Sequence
  Synthetic Polypeptide
  MISC_FEATURE (2)..(2): Allylglycine
  MISC_FEATURE (8)..(8): Allylglycine
FXNINNVXNF

SEQ ID NO: 31 - protein, 10 aa, Artificial Sequence
  Synthetic Polypeptide
FVNINNVVNF

SEQ ID NO: 32 - protein, 10 aa, Artificial Sequence
  Synthetic Polypeptide
FIDINDVINF

SEQ ID NO: 33 - protein, 10 aa, Artificial Sequence
  Synthetic Polypeptide
FIDINDVINW

SEQ ID NO: 34 - protein, 9 aa, Artificial Sequence
  Synthetic Polypeptide
FTDINDVTN

SEQ ID NO: 35 - protein, 10 aa, Artificial Sequence
  Synthetic Polypeptide
  MISC_FEATURE (2)..(2): 2-Aminobutyric acid
  MISC_FEATURE (8)..(8): 2-Aminobutyric acid
AXNINNVXNF

SEQ ID NO: 36 - protein, 10 aa, Artificial Sequence
  Synthetic Polypeptide
  MISC_FEATURE (1)..(1): 4-chlorophenylalanine
  MISC_FEATURE (2)..(2): 2-Aminobutyric acid
  MISC_FEATURE (8)..(8): 2-Aminobutyric acid
XXNINNVXNF